(19) United States
(12) Patent Application Publication
(10) Pub. No.: US 2011/0190586 A1
(43) Pub. Date: Aug. 4, 2011

(54) METHODS AND SYSTEMS FOR INTRAVASCULAR IMAGING AND FLUSHING

(75) Inventor: Nathaniel J. Kemp, Concord, MA (US)

(73) Assignee: Volcano Corporation, San Diego, CA (US)

(21) Appl. No.: 12/981,115

(22) Filed: Dec. 29, 2010

Related U.S. Application Data
(63) Continuation-in-part of application No. 12/892,229, filed on Sep. 28, 2010.
Application No. 12/892,229 is a continuation of application No. PCT/US2009/038832, filed on Mar. 30, 2009.
(60) Provisional application No. 61/040,630, filed on Mar. 28, 2008.

(51) Int. Cl. A61B 1/12

(52) U.S. Cl. 600/156

(57) ABSTRACT

A method and apparatus for initiating intravascular imaging and flushing is described herein.

### CROSS-REFERENCE TO RELATED APPLICATIONS

This application is a continuation-in-part application from U.S. patent application Ser. No. 12/892,229, which was filed Sep. 28, 2010 and which claims priority to PCT Patent Application No. PCT/US2009/038832, which was filed on Mar. 30, 2009, and which claims priority to U.S. Provisional Application Ser. No. 61/040,630, filed Mar. 28, 2008, all incorporated by reference herein.

### BACKGROUND OF THE INVENTION

The field of the invention is a process and apparatus for imaging, and more particularly to intravascular imaging.

Efforts have been made to initiate imaging systems, although problems arise with flushing and presence of blood. The present invention attempts to solve this problem, as well as others.

### SUMMARY OF THE INVENTION

A method and apparatus for coordinating the intravascular imaging and flushing is described herein. In one embodiment, the method for starting the longitudinal motion of a catheter system comprises starting a flushing sequence; detecting if blood is present; and initiating the longitudinal motion of the catheter. In one embodiment, the method for detecting the start and stop of a flush during a catheter imaging procedure comprises: reducing the total volume-per-bolus of blood-clearing fluid delivered during an imaging step; acquiring an image frame; and detecting if flushing is occurring.

In another embodiment, a method for detecting the start and stop of a flush during a catheter imaging procedure comprises coupling an imaging system in communication with an electro-mechanically controlled syringe pump; pre-loading the syringe reservoir with a clearing fluid; and injecting the clearing fluid into the distal end of a catheter only as requested by the imaging system. In another embodiment, a method for detecting the start and stop of a flush during a catheter imaging procedure comprises positioning an imaging transducer in a fixed longitudinal position and continuously acquiring images with an imaging system; analyzing the incoming images and determining if the vessel is cleared or not cleared of blood; and initiating the longitudinal translation of the imaging transducer when a cleared vessel is detected.

In another embodiment, a catheter system for the initiation of imaging comprises a flushing apparatus operably coupled to the distal end of a catheter; an imaging system operably coupled to the distal end of the catheter to detect if blood is present; and a longitudinal displacement apparatus operably coupled to the catheter to initiate the longitudinal motion of the catheter upon the detection of blood.

The foregoing and other features and advantages of the invention are apparent from the following detailed description of exemplary embodiments, read in conjunction with the accompanying drawings. The detailed description and drawings are merely illustrative of the invention rather than limiting, the scope of the invention being defined by the appended claims and equivalents thereof.

### BRIEF DESCRIPTION OF THE FIGURES

FIG. 1A is a schematic view of one embodiment of the simultaneous OCT measurement and hemoglobin reflectivity system 100; FIG. 1B is a schematic view of another embodiment of the simultaneous OCT measurement and hemoglobin reflectivity system 100; and FIG. 1C is a schematic view of another embodiment of the Simultaneous OCT measurement and hemoglobin reflectivity system 100, where “PBS” is Polarizing Beam Splitter, “Hb” is Hemoglobin, and “WDM” is Wavelength Division Multiplexer.

FIG. 2 is an OCT image of a coronary vessel with white thrombus.

FIG. 3A is a schematic view of one embodiment of the simultaneous thrombus visualization and laser thrombolysis system 300; FIG. 3B is a schematic of another embodiment of the simultaneous thrombus visualization and laser thrombolysis system 300; and FIG. 3C is a schematic of another embodiment of the simultaneous thrombus visualization and laser thrombolysis system 300.

FIG. 4A is a schematic of an exemplary thrombus laser, where G1, G2, are fiber Bragg gratings, M is an amplitude modulator, P is a fiber polarizer, LDs are laser diodes, and PM is polarization maintaining; and FIG. 4B is a schematic of an exemplary FOPA.

FIG. 5 is a flow chart of the thrombosis detection and treatment sequence.

FIG. 6A is a flow chart of the initiation sequence, in accordance with one embodiment;

FIG. 6B is a flow chart of the flushing sequence, in accordance with one embodiment; FIG. 6C is a flow chart of the flushing sequence, in accordance with one embodiment; FIG. 6D is a flow chart of the flushing sequence, in accordance with one embodiment; and FIG. 6E is a flow chart of the flushing sequence, in accordance with one embodiment.

FIGS. 7A and B are the amplitude and phase data, where FIG. 7A is saline and FIG. 7B is showing a maximum temperature increase of 18.6 degrees C. of metallic nanoparticles during 2 seconds of 532 nm laser heating with a 10 Hz modulation frequency and a power 400 mW.

FIG. 8 is the oxygenated hemoglobin (“HbO₂”) and deoxygenated hemoglobin (“Hb”) absorption spectrum compared water.

### DETAILED DESCRIPTION OF THE PREFERRED EMBODIMENTS

The systems and methods of use described herein may be embodied in many different forms and should not be construed as limited to the embodiments set forth herein. Accordingly, the systems and methods of use described herein may take the form of an entirely hardware embodiment, an entirely software embodiment or an embodiment combining software and hardware aspects. The systems and methods of use described herein can be performed using any type of computing device, such as a computer, that includes a processor or any combination of computing devices where each device performs at least part of the process or method.

Suitable computing devices typically include mass memory and typically include communication between devices. The mass memory illustrates a type of computer-readable media, namely computer storage media. Computer storage media may include volatile, nonvolatile, removable, and non-removable media implemented in any method or technology for storage of information, such as computer readable instructions, data structures, program modules, or other data. Examples of computer storage media include RAM, ROM, EEPROM, flash memory, or other memory technology, CD-ROM, digital versatile disks (DVD) or other optical storage, magnetic cassettes, magnetic tape, magnetic disk storage or other magnetic storage devices, Radiofrequency Identification tags or chips, or any other medium which can be used to store the desired information and which can be accessed by a computing device.

Methods of communication between devices or components of a system can include both wired and wireless (e.g., RF, optical, or infrared) communications methods and such methods provide another type of computer readable media; namely communication media. Communication media typically embodies computer-readable instructions, data structures, program modules, or other data in a modulated data signal such as a carrier wave, data signal, or other transport mechanism and include any information delivery media. The terms “modulated data signal,” and “carrier-wave signal” includes a signal that has one or more of its characteristics set or changed in such a manner as to encode information, instructions, data, and the like, in the signal. By way of example, communication media includes wired media such as twisted pair, coaxial cable, fiber optics, wave guides, and other wired media and wireless media such as acoustic, RF, infrared, and other wireless media.

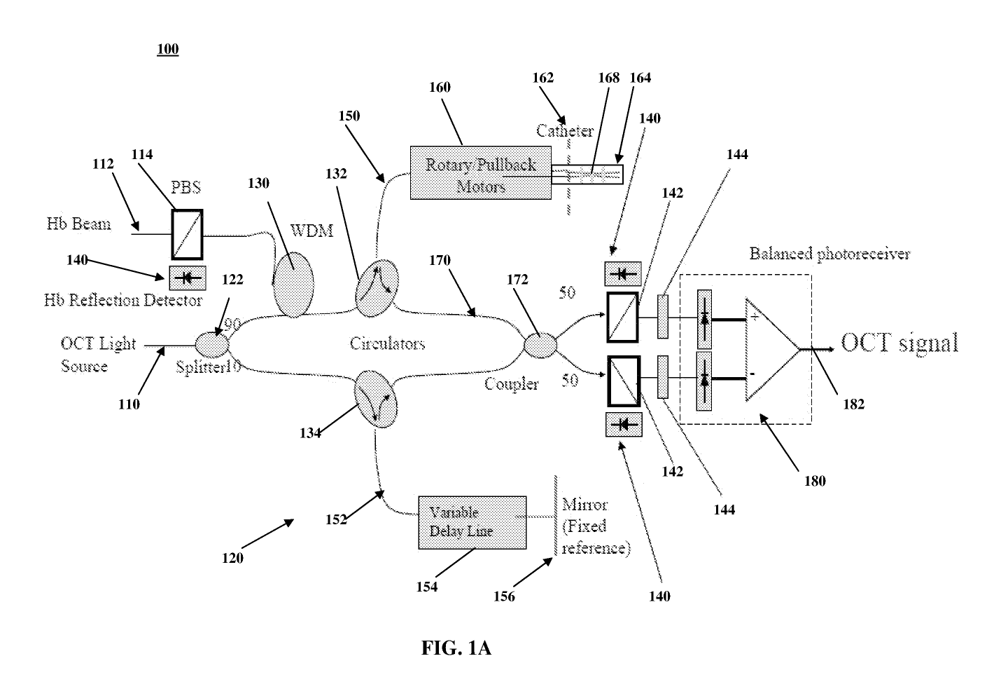

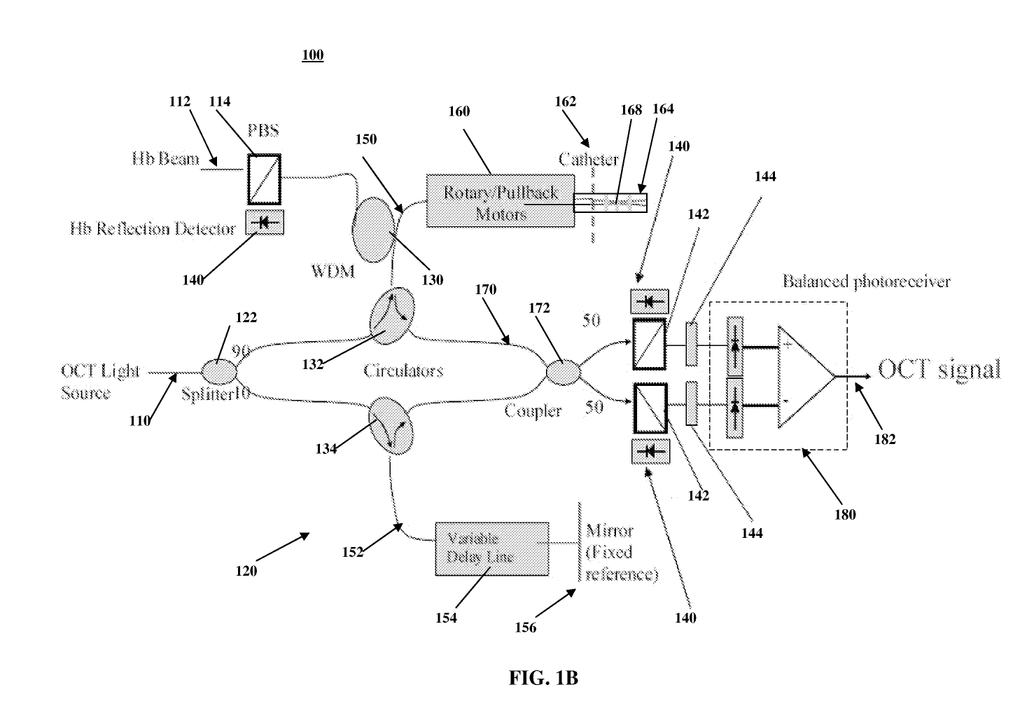

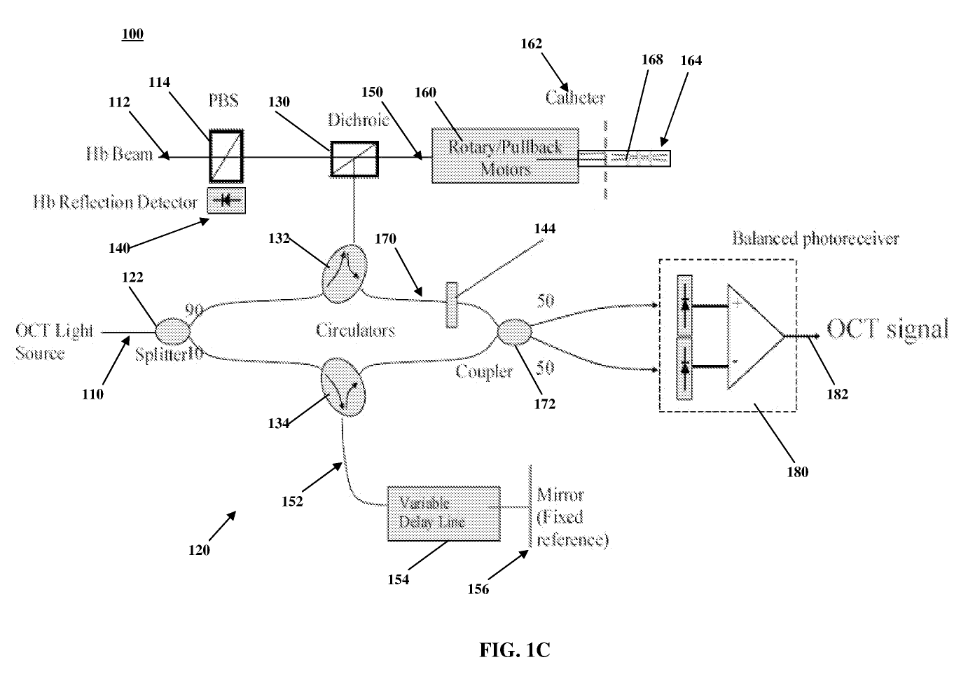

Generally speaking, the method and apparatus for performing simultaneously an OCT scan of a blood vessel and a site-to-site measurement of hemoglobin reflectivity 100 are shown in FIG. 1A. Hemoglobin reflectivity may include it blood backscattering or erythrocyte backscattering. The simultaneous OCT measurement and hemoglobin reflectivity system 100 comprises using a first optical energy 110 and a second optical energy 112, wherein the first optical energy 110 is an OCT optical energy and the second optical energy 112 is selected for hemoglobin reflectivity and measurement. The first optical energy 110 allows for simultaneous OCT images of a blood vessel as well as detection of hemoglobin by the second optical energy 112 are on a site-to-site basis. In one embodiment, the hemoglobin reflectivity measurement is not depth resolved as compared to the OCT B-scan image, which is depth resolved. The hemoglobin reflectivity measurement provides a relative measure of hemoglobin concentration at each lateral location of the OCT image. The hemoglobin reflectivity measurement discriminates between red thrombus, white thrombus, and mixed thrombus, and the ability to treat the red thrombus, white thrombus, and mixed thrombus with radiant optical energy. For example, the optical absorption spectrum of oxygenated and deoxygenated hemoglobin may be targeted by selecting the wavelength(s) of the second optical energy to have a high, medium or low absorption by hemoglobin. For example, the 532 nm wavelength is absorbed by hemoglobin relatively strongly and reflection of this light from white thrombus would be higher than red thrombus. Additionally, the hemoglobin reflectivity detects the arrival and exit of a blood-clearing flush bolus, such as saline, contrast-agent, dextrose, index-matching reagent, and the like, which is delivered for OCT image collection.

The apparatus for simultaneous OCT measurement and hemoglobin reflectivity 100 is derived from any OCT system for imaging coronary arteries comprising a light source, which when incident on hemoglobin provides a source of contrast, wherein the light source may be such that the reflection from hemoglobin is substantially greater or less than light reflected from the luminal surface of a blood vessel. The OCT system may include a Fourier domain OCT (“FD-OCT”), sometimes known as Spectral Domain OCT (“SD-OCT”), or a Time-Domain OCT scanning (“TD-OCT”), where the optical path length of light in the reference arm of the interferometer is rapidly scanned over a distance corresponding to the imaging depth range. The OCT systems may be polarization-sensitive or phase-sensitive and adjusted accordingly.

The present methods, systems, and apparatuses may also be applied to other imaging systems, such as spectroscopic devices, (including fluorescence, absorption, scattering, and Raman spectroscopies), intravascular ultrasound (IVUS), Forward-Looking IVUS (FLIVUS), high intensity focused ultrasound (HIFU), radiofrequency, thermal imaging or thermography, optical light-based imaging, magnetic resonance, radiography, nuclear imaging, photoacoustic imaging, electrical impedance tomography, elastography, pressure sensing wires, intracardiac echocardiography (ICE), forward looking ICE and orthopedic, spinal imaging and neurological imaging, image guided therapeutic devices or therapeutic delivery devices, diagnostic delivery devices, and the like, which may utilize the embodiments described herein.

As shown in FIG. 1A, the apparatus comprises any OCT system 120 for imaging comprising: (1) a second optical energy 112 for hemoglobin and incident on hemoglobin (“Hemoglobin beam(s)” or “Hb beam”) to provide a source of contrast, wherein the hemoglobin beam(s) may be such that the reflection from hemoglobin is substantially greater or less than optical energy reflected from tissues not containing hemoglobin; (2) a bulk dichroic beamsplitter or wavelength division multiplexer (“WDM”) 130 for introducing the Hemoglobin beam 112 into the OCT optical interferometer system 120; (3) at least one optical detector 140 for measuring the backreflected Hemoglobin beam (Hb) light 168 from an imaged specimen 164; (4) at least one optical filter 144 that substantially blocks the Hemoglobin beam from entering detectors dedicated to OCT imaging; and (5) at least one dichroic filter 142 that separates the Hemoglobin beam from OCT beam and allows detection of Hemoglobin beam light reflected from the dichroic filters. For FIG. 1A, a detection path 170 is coupled to a 50/50 coupler 172, which is coupled to the dichroic filters 142 and the optical filters 144. A balanced photoreceiver 180 processes an OCT signal 182 to produce an OCT image through computer processors (not shown). Depending of the detector type used for the OCT system (photoreceiver, CCD, and the like), one or more optical filters 144 may be used to substantially block the Hb reflected light from entering the OCT detectors or photoreceivers 180. In one embodiment, the system for detecting backreflected HB light 100 at the HB light source with the detector 140 includes a non-ideal circulator 132. The non-ideal circulator 132 works at a wavelength different than the OCT source 110 wavelength. The optical filter prevents the entry of substantially all the Hb light into the OCT detector, where at least some small fraction of Hb beam photons will be incident on the OCT detectors.

In one embodiment and as shown in FIG. 1A, the OCT interferometer is a Mach-Zehnder interferometer configuration in a SS-OCT implementation, which measures the complex mutual coherence function (magnitude and phase) between two non-reciprocal optical paths, one sample path 150 encompassing an object under test 168 (i.e. “the specimen”) and the other to a reference path 152. An exemplary SS-OCT system is described in U.S. patent application Ser. No. 12/172,980, and incorporated by reference herein. Other interferometers are possible in alternative embodiments, such as Michelson and Common Path phase-sensitive interferometers, which can be configured to complete the Hemoglobin reflectivity measurement as well. The OCT interferometer comprises a light source for OCT emitting the first optical energy 110. The OCT light source may be a swept laser source such as a High Speed Scanning Laser HSL-2000 (Santec) with an instantaneous coherence length of over 10 mm. Alternatively, the light source includes a tunable laser, tunable-superluminescent diode (SLED) or other tunable source of photons. The swept laser source includes emitted light with a mean frequency of the output spectrum that varies over time. The OCT light source is coupled to a splitter 122, splitting the OCT light source 90% into the primary OCT interferometer and 10% into the an auxiliary wavemeter and an optical trigger generator for clocking the swept light source in order for providing an external clock signal to a high speed digitizer 270, as disclosed in commonly assigned application Ser. No. 12/173,004, filed Jul. 14, 2008, herein incorporated by reference. The splitter 122 splits the OCT light source 90% directed to port 1 of a 3-port optical circulator 132 for the sample path 150 and 10% of the light is directed to port 1 of a 3-port optical circulator 134 for the reference path 152. The reference path 152 includes a variable delay line 154 and a minor 156 to provide a fixed reference. In one embodiment, port 2 of circulator 132 for the sample path 150 is coupled to a Rotary/Pullback Motor 160 and to a probe or catheter 162 to reflect light off the specimen 164. In one example, the specimen 164 may be a blood vessel, an artery such as a coronary artery, a cerebral artery, peripheral artery, a pulmonary artery, venous vessels, or any other vessels, lumen, and the like including hemoglobin. The probe or catheter 162 is coupled to the Rotary/Pullback Motor 160 via a Fiber Optic Rotary Junction (“FORJ”). Examples of a rotating catheter tip for the sample path include, a catheter for in vivo imaging as described in U.S. patent application Ser. No. 12/172,922, filed Jul. 14, 2008; a rotating optical catheter tip as described in U.S. patent application Ser. No. 11/551,684; or a rotating catheter probe as described in U.S. patent application Ser. No. 11/551,684; each herein incorporated by reference for the methods, apparatuses and systems taught therein. The catheter can be located within a subject to allow light reflection off of subject tissues to obtain optical measurements, medical diagnosis, treatment, and the like.

In operation, the apparatus couples the Hemoglobin beam light source (“Hb beam” or “HB light”) into the OCT interferometer 120 so that Hemoglobin beam light 112 enters the sample path 150 of the interferometer and is incident on the specimen 164 being imaged, as shown in FIG. 1A. In one embodiment, the Hemoglobin beam light 112 passes through a polarization beam splitter (PBS) 112. The PBS is one method to achieve better isolation between the incident and back reflected Hb light. By using the PBS, polarized light from the Hb laser is all coupled into the fiber (with none reflected). If a standard Beam Splitter (BS) is used (non-polarizing) then some of the incident light will be lost due to reflection in the BS. If a BS is used then a beam block should be placed in the path of the light reflected from the BS on entrance. The Hemoglobin beam light source emits at least some wavelength that provides contrast for hemoglobin. The contrast may be provided by either increased absorption or increased scattering of at least one wavelength emitted by the light source. Light wavelengths that provide increased absorption by oxygenated or deoxygenated hemoglobin are 532 nm, but may also include wavelengths in the range of 530-540 nm. If more than one wavelength is used to detect hemoglobin, then the intensity of reflected light for the two wavelengths can be processed differentially.

The component for coupling the Hemoglobin beam is an optical element 130 that provides for the simultaneous transmission of the Hemoglobin beam light and the OCT light beam. The optical element 130 may be either a wavelength division multiplexer (“WDM”), a fiber based WDM, or a dichroic filter such as an optical filter element, as shown in FIG. 1C. A WDM 130 is shown in FIG. 1A as the component for coupling the Hemoglobin beam and simultaneous transmission of the OCT light beam. The Hemoglobin beam may be introduced into either the source path of the OCT interferometer or into the sample path 150 of the OCT interferometer 120, as shown in FIG. 1B.

The Hemoglobin beam light reflects from a specimen 164, which includes a hemoglobin sample and returns to the OCT interferometer system 120. The reflected Hemoglobin light may be coupled out of the OCT interferometer in the sample path 150 of the OCT interferometer 120, or in a detection path 170 of the OCT interferometer 120, as shown in FIG. 1A, or in both sample 150 and detection paths 170 of the OCT interferometer. The selection of the location at which to couple in/out Hemoglobin light from the OCT interferometer 120 depends on the spectral transmission characteristics of the fiber circulator and couplers.

The reflected Hemoglobin beam light is directed into the optical detector 140, which may be a photovoltaic detector 140 that is sensitive to Hemoglobin beam light. Generally speaking, the Hb detector 140 coupled to the Hb light source 112 is not required as long as at least one Hb detector 140 is in the detection path 170. The Hb light 168 that returns from the specimen 164 only couples back to the Hb source path because the circulator is designed to operate at the OCT source wavelength and not necessarily the Hb wavelength. In these cases when the OCT source and Hb wavelengths are different, some Hb light reflected from the specimen (e.g., blood vessel) can couple into the entrance port of the circulator. In most implementations, the Hb detector 140 associated with the Hb source light 112 can removed and one Hb detector 140 in the detection path 170 may be implemented. The reflected Hemoglobin beam may be directed into the photovoltaic detector using either a dichroic filter 142 or a wavelength division multiplexer. The optical element 130 to direct Hemoglobin light into and out of the OCT interferometer 120 could be located before the circulator 132 into port 1 of the circulator, as shown in FIG. 1A, or after the circulator 132 into port 2 of the circulator in the sample path 150 but before the fiber optic rotary junction (“FORJ”). The photovoltaic detector 140 may be set to receive the reflected Hemoglobin beam or sensitivity to the wavelength of light of the Hemoglobin beam. Alternatively, the reflected hemoglobin beam may be received by the photovoltaic detector 140 before returning to the detection path 170 of the OCT interferometer, as shown in FIG. 1C. The dichroic beamsplitter 130 replaces the WDM and separates the reflected Hemoglobin beam and directs it to the Hemoglobin reflection detector 140. The Hemoglobin beam blocking OCT transmission 144 may also be placed in the detection arm 170 after the circulator 132 and before the 50/50 coupler 172, as shown in FIG. 1C. The detectors may be in communication with the computer components via digital communication (electrical, digital optical, or wireless; parallel or serial data transmission; computer data bus) or analog. Communication between any proximal and distal end of any party of the system, device, or apparatus may be by any communication devices, such as wires, optics, wireless, RF, and the like.

In one embodiment, the measurement of the reflected Hemoglobin beam is completed for each OCT A-scan. The reflected Hemoglobin beam that is detected or measured for each A-scan may be integrated over various time periods of the OCT scan. In another embodiment, the reflected Hemoglobin beam is measured over the time period between OCT A-scans, where the time period between OCT A-scans is when the OCT source light is off and the interference fringes are not being recorded. In this embodiment of recording Hemoglobin reflected light between OCT A-scans, the Hemoglobin light does not introduce additional noise into the detection circuitry for the OCT signal. Alternatively, if the Hemoglobin light is recorded over a time period that overlaps with the OCT A-scan, then measures are taken to limit or substantially eliminate any Hemoglobin light from entering the OCT detectors while allowing the OCT light to enter the OCT detectors. In this embodiment, the optical dichroic filters may be used to separate OCT source light and Hemoglobin reflected light into physically distinct circuitry. However, if the wavelength of light used for the Hemoglobin beam and the OCT beam are substantially different, then the detectors' spectral sensitivity may be set to different spectral sensitivities as to not require additional filtering.

Therefore, the Hemoglobin reflection measurement may be performed either concomitantly with the OCT A-scan or between successive OCT A-scans when OCT detection circuitry is not recording signals. In one embodiment, the Hemoglobin reflection measurement is between OCT A-scans and does not interfere with OCT measurement in any way. In another embodiment, hemoglobin and OCT measurements partially overlap. And in one embodiment, Hemoglobin measurement is performed during the OCT A-scan. In another embodiment, the Hemoglobin reflection measurement is performed at any time and the OCT measurement may be disregarded.

The reflected Hemoglobin light beam provides a relative measure of Hemoglobin light at each lateral imaging position. When the Hemoglobin beam is strongly absorbed by Hemoglobin, a relatively weak reflected Hemoglobin signal indicates the presence of Hemoglobin. Alternatively, if the Hemoglobin beam is strongly backscattered by Hemoglobin, a relatively weak signal represents the absence or low levels of Hemoglobin. In one embodiment, if the hemoglobin beam has an optical wavelength of 532 nm, hemoglobin absorbs this 532 nm wavelength strongly and a low signal amplitude represents presence of hemoglobin while a large signal amplitude may represent the presence of white thrombus.

Other causes might be responsible for the strong or weak Hemoglobin signal. For example, the Hemoglobin signal may be weak because flowing blood is absorbing the Hemoglobin beam rather than red thrombus being present. Thus, the OCT image may first identify presence of a thrombus, and subsequently the Hemoglobin beam determines the Hemoglobin content. Likewise, the Hemoglobin signal may be weak because the Hemoglobin beam is not focused well on the thrombus, which could make white thrombus appear as red thrombus due to the lower Hemoglobin signal, even though it's lower due to other effects and not lower due to Hemoglobin absorption. In this case, the OCT signal/image is first used to gauge how those other effects will change the relative values of the Hemoglobin signal before making a determination of the Hemoglobin content. Measuring the Hemoglobin content based on the Hemoglobin signal reflectivity is refined with prior knowledge from the OCT image.

In another embodiment, two or more Hemoglobin signals with different wavelengths could provide additional (differential) discrimination of the Hemoglobin content. The algorithm to decide Hemoglobin content should include both the two or more Hemoglobin signal beams and also the exponential nature of the OCT intensity decrease vs. depth, which are complimentary because the OCT signal provides a measure of scattering and the Hb signal provides a measure of absorption. Using both should provide better sensitivity than one measurement alone.

Thrombus Detection and Treatment

Ambiguity may arise in detected hemoglobin reflected signal intensity due to effects such as blood in the image field, imperfect focusing distance from tissue to catheter, or other. However, these effects will also be apparent in the OCT image. Thus, the OCT image can be used to determine the structural characteristics in the image and to detect whether thrombus is present, and then the Hemoglobin measurement can be employed to detect the Hemoglobin reflectivity at the location of interest. A matrix may help in differentiating white from red thrombus. For each type of thrombus, there is reflectivity of OCT light and the Hb light. For white thrombus, the OCT light scatters less strongly in white thrombus so that the vessel wall behind the thrombus is brighter for white thrombus compared to red thrombus. The OCT signal distinguishes between red and white thrombus based on the relative attenuation of the OCT light through the white thrombus. The Hb light will be more strongly backreflected from white thrombus than red thrombus. The white thrombus will not absorb the HB light and thus give rise to a larger backreflected signal of the Hb light. For red thrombus, the OCT light is more strongly attenuated by red thrombus than white thrombus. By examining the attenuation of OCT light through the thrombus, an estimate can be made whether the thrombus is red or white. The Hb light will be strongly absorbed by red thrombus and thus the magnitude of Hb light backscattered from the red thrombus will be less. By combining the OCT attenuation and HB reflectivity, a specific estimate if the thrombus is red or white may be obtained, where white corresponds to relatively low OCT attenuation and relatively high Hb reflectivity and red corresponds to relatively high OCT attenuation and low Hb reflectivity.

The thrombosis may be present in the coronary arteries, cerebral arteries, peripheral arteries, pulmonary arteries, venous vessels, or any other vessels subjected to thrombosis. The mechanical properties of the thrombus change with age, so fresh thrombus is mechanically softer and more pliable than older thrombus, which becomes harder and less pliable. Because older thrombus is less pliable and more rigid, risk of an infarction is greater with old thrombus that is mechanically dislodged from the vessel lumen. For these reasons, thrombolysis of older thrombus is especially important and targeting of this older thrombus can be accomplished by the method and apparatus allows simultaneous visualization of the intravascular thrombus while conducting laser thrombolysis. The more rigid thrombus may be detected by birefringence, polarization-sensitive OCT, which more readily understood by nonprovisional application entitled “Fiber-Based Single Channel Polarization-Sensitive Spectral Interferometry”, U.S. Ser. No. 12/131,825, filed Jun. 2, 2008, incorporated by reference herein. Additionally, differences in the thrombus, such as the amount of platelets or concentration of hemoglobin may be determined by the OCT imaging and hemoglobin beam. The intracellular components may also be discerned depending signal intensity, polarization state, birefringence, and phase-sensitive information obtained and described herein.

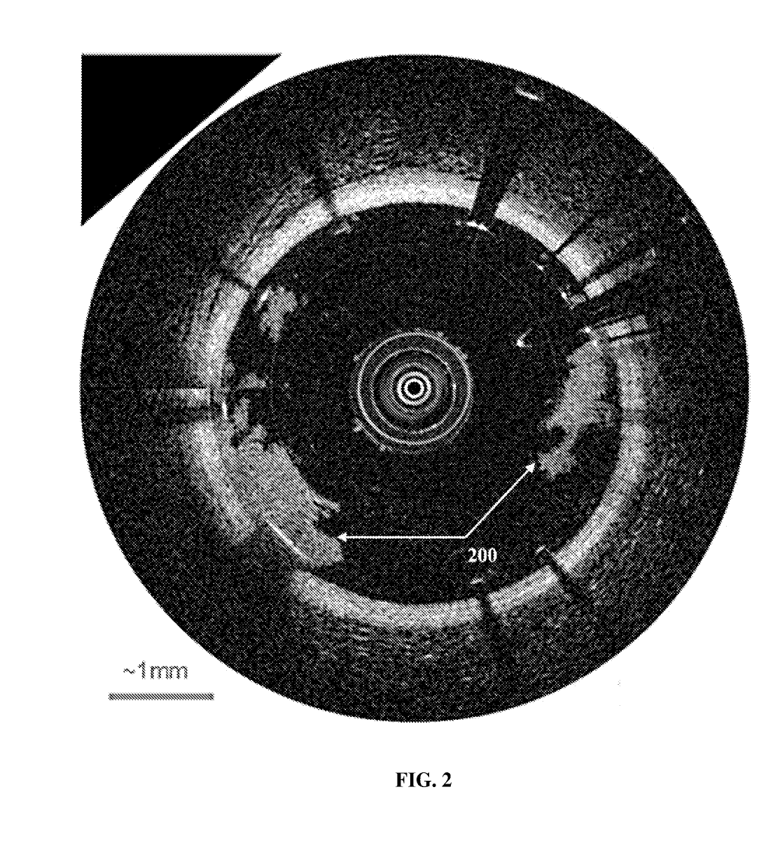

An OCT image of white thrombus 200 is shown in FIG. 2. The Hemoglobin measurement may also detect white thrombus 200 or red thrombus and discriminate between the two thrombi either through signal attenuation of one or more wavelengths. Thrombus color may be differentiated by the OCT image, where the white thrombi do not cast shadows like red thrombi, or the thrombus may be identified either by the physician or using a feature identification algorithm. The exponential nature of the OCT intensity decrease vs. depth differentiates the red vs. white thrombus color. As shown in FIG. 2, white thrombus includes signal rich and low-backscattering projections; alternatively, red thrombus includes signal high-backscattering protrusions with signal-free shadowing. FIG. 2 was obtained from a live pig using intravascular OCT under general anesthesia, and a heart catheterization procedure was performed similar to for a human patient. A guiding catheter may engage a blood vessel, and a wire was passed in a coronary artery. Over the wire, the OCT catheter may be inserted into the vessel, and during a flush of saline or contrast or both, the coronary artery is cleared to image the vessel wall. Image construction software generated an image of the coronary artery from the reflected OCT beam light and performed a time-frequency transform (e.g. Fourier transform) on the light signal data generating amplitude and phase data. The amplitude and phase data (optical path length difference (cτ) or optical time-delay (τ)) can be separated into discrete channels and a plot of intensity vs. depth (or amplitude vs. depth) can be generated for each channel. Such a plot is known in the art as an “A” scan. The composition of all the “A” scans can comprise one image.

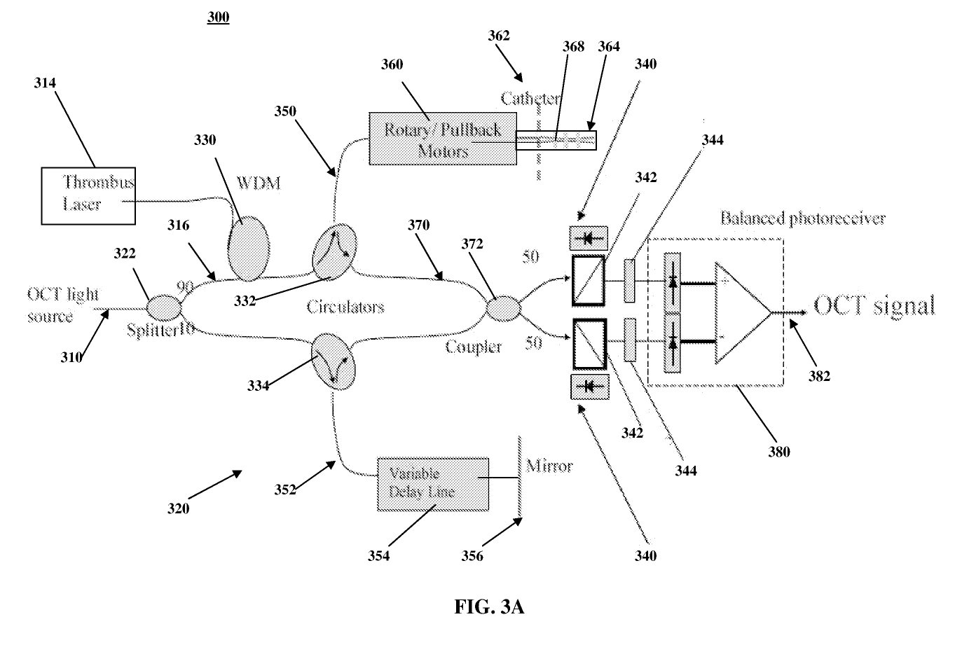

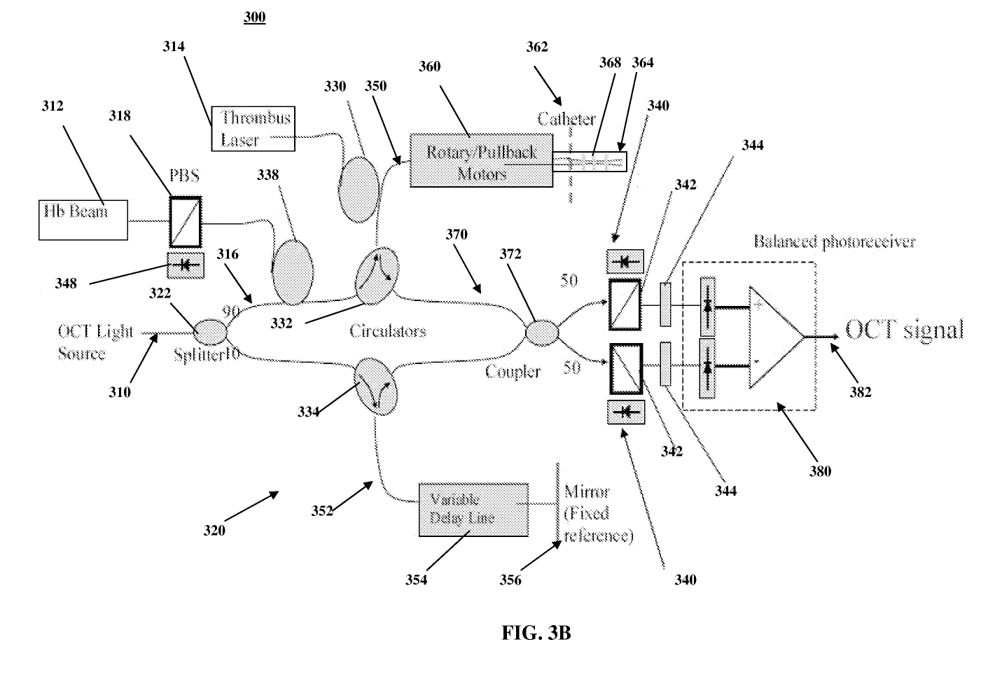

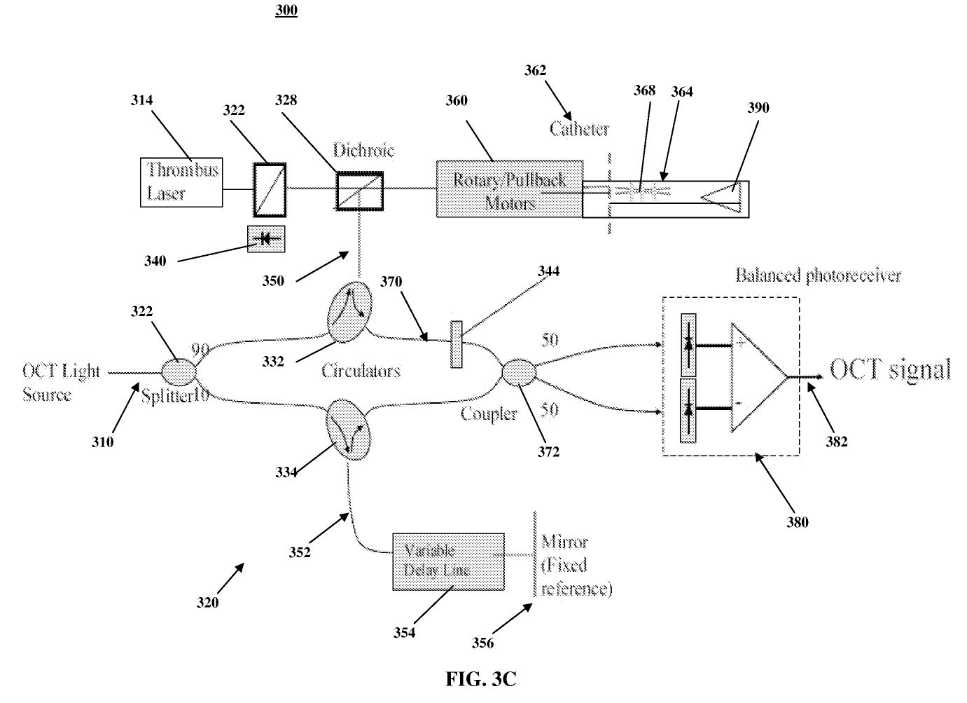

The method and apparatus allows simultaneous visualization of the intravascular thrombus while conducting laser thrombolysis 300, as shown in FIG. 3A. In one embodiment, the simultaneous thrombus visualization and laser thrombolysis system 300 comprises a thrombus laser source 314 is coupled to an OCT interferometer system 320 by a Wavelength Division Multiplexer 330 in a source path 316, as shown in FIG. 3A. As indicated previously, the OCT system 320 includes an OCT optical energy 312 coupled to a 90/10 splitter 322. The splitter 322 splits the OCT light source 90% directed to port 1 of a 3-port optical circulator 332 for a sample path 350 and 10% of the light is directed to port 1 of a 3-port optical circulator 334 for the reference path 352. The reference path 152 includes a variable delay line 354 and a minor 356 to provide a fixed reference. In one embodiment, port 2 of circulator 332 for the sample path 150 is coupled to a Rotary/Pullback Motor 360 and to a probe or catheter 362 to reflect light off a specimen 364. A detection path 370 is coupled to a 50/50 coupler 372, which is coupled to at least one dichroic filter 342, at least one thrombus laser reflection detector 340, and at least one thrombus beam blocking OCT transmission filter 344 to receive a backreflected thrombus beam 368. A balanced photoreceiver 380 processes an OCT signal 382 to produce an OCT image of the white or red thrombi through computer processors (not shown). Subsequently, the thrombus laser source 314 is activated and coupled to the white or red thrombi through the WDM 330 in the OCT system 320 to lyse the white or red thrombi.

Alternatively, the thrombus laser source 314 is coupled to the OCT interferometer 320 in the sample arm 350 before the Rotary/Pullback Motors 360, as shown in FIG. 3B, where the thrombus laser 314 is coupled to the Wave Division Multiplexer 330 or a Y-cladding mode coupler monitor to couple the thrombus laser 314 to lyse the red or white thrombi in the specimen 364. The detection path 370 is coupled to the 50/50 coupler 372, which is coupled to at least one dichroic filter 342, at least one hemoglobin reflection detector/thrombus laser reflection detector 340, and at least one hemoglobin beam/thrombus beam blocking OCT transmission filter 344 to produce the OCT image via the OCT signal 382. Alternatively, a hemoglobin beam 312 may be coupled to the OCT system 320 through a PBS 318 and a WDM 338 in the source path 116. As such, a reflected hemoglobin beam 368 from the specimen 364 may be received by an hemoglobin photovoltaic detector 348 before returning to the detection path 370 of the OCT interferometer, as shown in FIG. 3B.

Alternatively, a dichroic beamsplitter 328 separates the reflected Hemoglobin beam 368 before entering the circulator 332 and directs it to the Hemoglobin reflection detector 340 through a PBS 322, as shown in FIG. 3C. The circulator 332 works non-ideally to back couple light from the specimen 364 at the HB reflected 368 wavelength. Only the beamsplitter 322 is active in being able to detect backreflected light from the thrombus. The beamsplitter 322 is positioned correctly to direct some of the Hb reflected beam 368 into the detector 340. The Hemoglobin beam blocking OCT transmission filter 344 may also be placed in the detection arm 370 after the circulator 332 and before the 50/50 coupler 372. The thrombus laser source 314 may be interchangeable with the Hb beam source 312, depending upon the application desired. However, it is to be understood that the thrombus laser source 314 may also provide for hemoglobin reflectivity, measurement, imaging, and detection as in the similar manner that the hemoglobin beam light source 312 conducts hemoglobin reflectivity, measurement, imaging, and detection.

The thrombus laser 314 can emit optical energy over a multiplicity of optical wavelengths, frequencies, and pulse durations to achieve the controlled heating of the red and white thrombi. The thrombus laser source 314 is further explained below. In one example, the heating of the thrombi with green light near the green spectrum can be used to cause ablation of the thrombus by light absorption by red blood cells and/or platelets located in the thrombi. In order to achieve heating of the thrombi and lyse the thrombi, the pulse duration is selected by the optical absorption length (δ) of light in the thrombus or the lateral spotsize of light incident on the thrombus (d). The mechanism of breaking the thrombus into smaller sized fragments is one by which light is absorbed by the thrombus; the absorbed light generates thermal injury in the thrombus that results in thermal elastic expansion of the thrombus material; the thrombus material that is mechanically damaged is fragmented into a piece of material. By continuing with this process the thrombus is fragmented into smaller parts or micron-sized pieces. The principle of selective photothermolysis can be used to specify the appropriate pulse duration for targeted particles or clusters of particles of a given size, which is further explained below.

Selective Photothermolysis

Selective pulsed laser photothermolysis can be used to heat the thrombi and selectively injure and/or kill these cells. By absorbing light energy, the thrombi or clusters of thrombi temperature increases and can induce explosive vaporization of a thin layer of fluid in contact with the thrombi, as to cause a microexplosion within the cell. A conventional vapor bubble can be created that expands on the nano-second timescale as the initial high vapor pressure overcomes the surface tension of the fluid. The expansion and collapse of bubbles can also cause a second shock wave that travels outward and interacts with the cell to disrupt the cellular membrane. Thrombi that have hemoglobin can be killed, while adjacent cells can remain viable. Additionally, the heating energy, for example, a pulsed laser light can be used to selectively heat the thrombi to induce apoptosis, protein inactivation through denaturation or coagulation of protein form increased temperature of the thrombi by the pulsed laser, or damage to specific cellular structures by the interaction of the heated thrombi and cellular structures.

A spatially localized temperature increase can be generated within individual macrophages or other cells when incident photons are absorbed by the thrombi. Spatially selective confinement can be accomplished by using laser dosimetry with a wavelength that is absorbed by the thrombi and pulse duration for spatial confinement within the macrophage or other cells. Selection of appropriate pulse duration can be used to allow application of the principle of selective photothermolysis so that temperature increase can be confined more to thrombi or been targeted by the thrombi. Neighboring cells not comprising the hemoglobin can be spared.

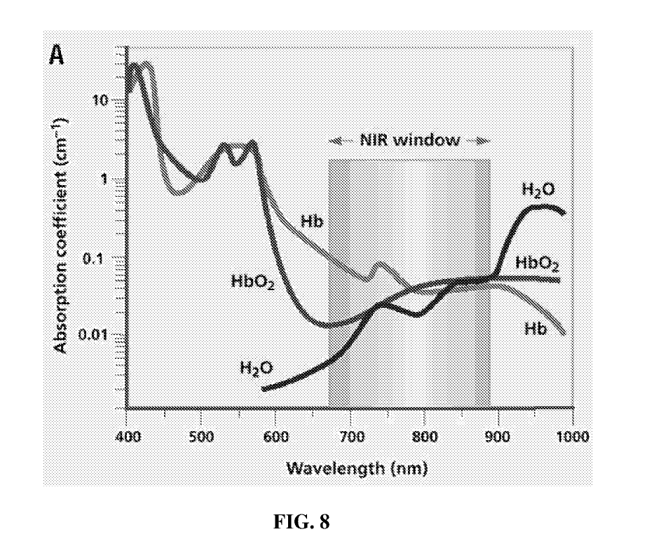

The principles of selective photothermolysis can be used to determine the proper lysing protocol or parameters. Using selective photothermolysis, four exemplary parameters that can be determined in selecting a killing protocol include wavelength of the energy source, dose (energy/area), pulse duration, and spot size. To select an appropriate wavelength, the absorption properties of the particle or cluster of particles and the cell and/or surrounding tissues can be determined. A wavelength of killing light energy can be selected to be more strongly absorbed by the particle than the cell or any surrounding tissue or any tissue or composition between the source and the particle. For example, the absorbance spectrum of fat, normal aortic tissue and oxygenated hemoglobin are known and nadir at about 700 nm. Although water has a nadir at about 500 nm, its absorbance is negligible at about 700 nm, and non-existent at 532, as shown in FIG. 8.

Thus, wavelength can be determined based on the absorption of the targeted particle and the absorption of other compositions in the subject, such as tissues, endogenous chromophores, protein composition, or any other absorptive characteristic of the subject imposed between the energy source and the target particle. The pulse duration can be determined by estimating the thermal relaxation time of the target particle. Thermal relaxation time can be based on the geometry of the particle and the diffusion of heat into media or tissue surrounding the target particle. An appropriate dose can also be determined. The dosage used can be related to the pulse duration. As pulse duration is lessened, the temperature used to kill a cell can be elevated. The change in temperature used for a given pulse duration for killing a cell can be determined by using the Aharenius damage integral, which is known to those skilled in the art. The spot size used can also be related to fluence. Thus, a desired spot size can be selected based on the desired fluence. Spot size can be selected to be approximately equal to the depth of the targeted cells.

A detectable internal strain field can be generated in the thrombi when a metallic composition, i.e. hemoglobin, is under the action of an external force or energy. The internal strain field can be detected using phase sensitive OCT using block correlation signal processing techniques that have been applied in elasticity imaging in ultrasound imaging. The external force may be provided by the application of the thrombus laser, i.e. a pulsed light source can be applied to the thrombi and a thermoelastic strain field can be detected with phase-sensitive OCT system, which can be readily understood by U.S. patent application Ser. No. 11/784,477, filed Nov. 8, 2007, and herein incorporated by reference. Action of the external force on each hemoglobin can produce movement of the hemoglobin (zₙₚ(t)) that produces a change in the cellular membrane tension level or an internal strain field within a cell. Action of a force on each hemoglobin in a thrombi or tissues produces a movement of the hemoglobin (zₙₚ(t)). Movement of the hemoglobin can be along the z-direction. The hemoglobin can also have movement in any direction that can be written as vector displacement, uₙₚ(rₒ) for a hemoglobin positioned at rₒ. Hemoglobin displacement uₙₚ(rₒ) can produce a displacement field (u(r,rₒ)) in the proteins in the thrombi containing the hemoglobin and surrounding cells. In the case of a homogeneous elastic media, the displacement field (u(r,rₒ)) can be computed for a semi-infinite half-space following, for example, the method of Mindlin (R. D. Mindlin, “A force at a point of a semi-infinite solid”, Physics 1936, 7:195-202, which is incorporated by reference for the methods taught therein). In the case of an inhomogeneous viscoelastic media, a finite element method numerical approach can be applied to compute the displacement field in the cell. The displacement field (u(r,rₒ)) produced by a hemoglobin positioned at rₒ can induce an internal stain field that is determined by change in the displacement field along a particular direction. The strain field (eεᵢⱼ(r,rₒ)) is a tensor quantity and is given by Equation (1),

$${ɛ}_{ij} ( r , {r}_{o} ) = \frac{\partial {u}_{i} ( r , {r}_{o} )}{\partial {x}_{j}} ; \qquad ( 1 )$$

where uᵢ(r,rₒ) is the i'th component of the displacement field and xⱼ is the j^{th} coordinate direction. For example, when j=3, x₃ is the z-direction. The internal strain field in thrombi due to all hemoglobins in the thrombi and surrounding thrombi is a superposition of the strain fields due to each hemoglobin. A detectable change in thrombi can also be caused with light energy. For example, pulsed laser light can be applied to contact the hemoglobin comprised by a thrombi. The application of light energy can cause a detectable change in optical path due to a change in optical refractive and thermal elastic expansion. The light energy can also cause motion of the cell, particle, or tissues proximate to the thrombi for detection by optical coherence tomography. Such movement can be caused by thermal elastic expansion. Alternatively, sound energy can motion of the cell, particle, or tissues proximate to the thrombi for detection by optical coherence tomography.

The change in strain field surrounding the thrombi can be detected using phase-sensitive optical coherence tomographic imaging modalities. In this approach phase sensitive interference fringes can be detected before and immediately after the application of a force on the hemoglobin. Utilizing block correlation algorithms for ultrasound elasticity imaging of spatially-resolved interference fringes recorded before and after application of a force on the hemoglobin particle can be used for determination of the spatially resolved strain field surrounding the cell. Thus, the thrombi can be detected by detecting the change in the thrombi caused by the interaction of the pulsed light energy causing a change in the thrombi with the hemoglobin using such a modality. The spatially resolved strain field due to application of the external force can be detected using a phase sensitive optical coherence tomographic imaging modality. Phase sensitive OCT imaging modalities can comprise a probe for transmitting and receiving light energy to and from the cell. The light energy used for OCT imaging modalities can be distinct from the light energy used to cause a change in the thrombi as would be clear to one skilled in the art. Thus, the OCT modality can use light energy for detection of the thrombi that is typical of OCT imaging systems. The systems described herein can also be used with a light source for causing a change in the cell. OCT imaging light energy can therefore be distinguished from light energy or energy that causes a change in the thrombi or thrombi changing energy. The probe can be sized, shaped and otherwise configured for intravascular operation. The probe can further comprise a magnetic source for applying the magnetic field to the cell. The magnetic field can be applied to the thrombi from a magnetic source located external to the subject or internal to the subject. The external source can be located in a probe or can be distinct from a probe. The external force can also be the application of pulsed laser light that is selectively absorbed by the hemoglobin of the thrombi and that generates a thermoelastic strain field surrounding the composition or particle. By recording images before or after pulsed laser exposure, the thermoelastic strain field in the tissue may be determined using block correlation algorithms applied for ultrasound elasticity and thermal imaging.

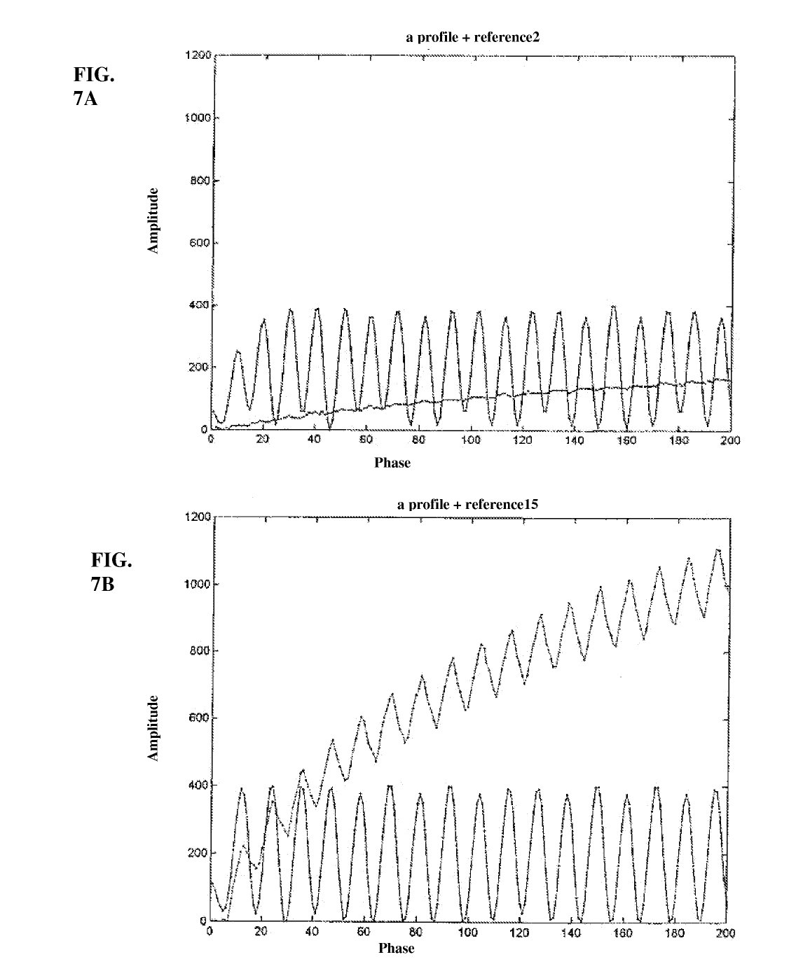

Atherosclerotic rabbit thoracic aorta injected with Iron Oxide Nanoparticles and saline 48 hours prior to imaging with optical coherence tomography and after injection. FIG. 7A shows maximum temperature increase of 2.9 degrees C. of saline during 2 seconds of 532 nm laser heating with a 10 Hz modulation frequency, 400 mW. FIGS. 7A and 7B is the Amplitude and Phase data, where FIG. 7B shows a maximum temperature increase of 18.6 degrees C. of metallic nanoparticles during 2 seconds of 532 nm laser heating with a 10 Hz modulation frequency and a power 400 mW the pulsed light can be in the green spectrum, preferably 532 nanometers a pulse duration of about 200 microseconds. The pulsed laser green light can cause a temperature increase of 18.6 degrees C., as shown in FIG. 7B. Higher temperature increased can also be achieved. For example, temperatures up to and greater than 40 degrees C. can be achieved. Pulsed laser light sources are discussed below, such as q-switched, free-running and femtosecond lasers and the like. Ultrashort-pulsed fiber lasers may be used, which demonstrate femtosecond passively mode-locked fiber oscillators by a variety of Ken-type saturable absorbers. Different wavelengths of light can be used to identify and heat the nanoparticles. Wavelength sensitivity of different nanoparticles can also enhance the specificity of heating endogenous tissue structures as to distinguish pathologic tissue structures from non-pathologic structures.

OCT temperature measurement may also be employed by recording an A-Scan or B-scan before and a second after pulsed laser excitation, where the OCT system is a phase sensitive OCT system. After recording scans before and after pulsed laser excitation, the interference fringe signals are correlated using a block correlation algorithm and then differentiated with respect to tissue depth to obtain a measure of the relative phase change due to the pulsed laser excitation. With a calibration of the combined thermo-refractive change and the thermo-elastic displacement, a depth resolved estimate of temperature resulting from the absorption of pulsed laser light is obtained Lipids in an atherosclerotic lesion might be more easily identified by an anomalous thermo-refractive and thermoelastic change.

Ablation Threshold

The ablation threshold of thrombus material is given by the partial vaporization theory. In this theory, light absorption leads to the generation of heat and rapid expansion of water to a vapor bubble. The rapid expansion of the vapor bubble leads to mechanical failure of the thrombi membrane and lysing of the thrombus in the region of light absorption. Ablation threshold may be predicted by the partial vaporization theory, which states that vaporization of water occurs when the temperature is raised to 100° C. The full energy of vaporization is not required before certain nucleation sites begin to form vapor bubbles. Thus the onset of ablation can be predicted by the following Equation (2):

$${E}_{th} = \frac{ρ c Δ {T}_{100}}{{μ}_{a}} ; \qquad ( 2 )$$

where Eₜₕ is the energy required to reach ablation threshold, ρ is the density, c is the specific heat, ΔT₁₀₀ is the number of degrees needed to reach 100° C., and μₐ is the absorption coefficient. This theory applies when the laser pulse is thermally confined, i.e., when the laser energy is deposited in the target absorber before the resultant heat has time to diffuse. Thermal confinement is achieved when the following Equation (3) is satisfied:

τₚ<τₜₕ;

where τₚ is the laser pulse duration, τₜₕ is the time of thermal confinement. Thermal confinement time may be limited by the absorption depth (δ) or the lateral spotsize (d) of light incident on the thrombus. The laser pulse duration (τₚ) is selected to be less that the thermal relaxation time. The thermal relaxation time (τₜₕ) relevant to selecting the laser pulse duration is the lesser of the longitudinal (δ²/4χ) or lateral thermal relaxation time (d²/16χ) where χ is the thermal diffusivity of the thrombus material (˜0.14 mm²/s).

Then according to the following table, the fluence rates (Power Density or Intensity) for Combined Thrombus Laser/OCT Thrombolysis can be calculated as shown in Table 1:

TABLE 1
Calculation of Fluence Rates (Power Density or Intensity)
for Combined Laser/OCT Thrombolysis

| | | | Pulse | | Power |
|---|---|---|---|---|---|
| Pulse | | Ablation | Energy | Fluence in | Density |
| Duration | Wavelength | Threshold | Used | SMF-28 | SMF-28 |
| (μs) | (nm) | (mJ/mm²) | (mJ) | (J/cm²) | (W/cm²) |
| 1 | 532 | 18 | 5 | 22918.31181 | 2.29E+10 |
| 100 | 532 | 17 | 17.5 | 21645.07226 | 2.16E+08 |
| 2000 | 532 | 15 | 5 | 19098.59317 | 9.55E+06 |
| 5000 | 532 | 15.5 | 5 | 19735.21294 | 3.95E+06 |
| 10000 | 532 | 12.5 | 5 | 15915.49431 | 1.59E+06 |

The last column represents the power density in a silica optical fiber with a 10 micron core diameter—similar to the Single Mode Fiber-28 (“SMF-28 fiber”) used for the OCT system for the pulse energy in the fourth column. A safe threshold for staying clear of damage in silica fibers is: 5×10⁸ W/cm². Based on the calculation in the Table 1 above, the pulse duration is at least about 100 μs or longer. When this pulse duration is longer than one OCT A-scan, a complete A-scan cannot be recorded between the pulsed laser irradiation because the ablating pulse is longer than one A-scan. In these cases the OCT imaging and laser thrombolysis may have to be performed at alternating times

If the spot diameter of focused OCT light on the luminal wall is assumed to be 50 μm or is 1.96×10⁻³ mm², then the energies required at the ablation threshold can be computed, as shown in Table 2:

TABLE 2
Ablation Threshold

| | Area (mm²) | |
|---|---|---|
| Ablation Threshold | for Spot Size of | Energy in mJ for Threshold |
| (mJ/mm²) | 50 μm | Ablation |
| 18 | 0.00196 | 0.03528 |
| 17 | 0.00196 | 0.03332 |
| 15 | 0.00196 | 0.0294 |
| 15.5 | 0.00196 | 0.03038 |
| 12.5 | 0.00196 | 0.0245 |

For each pulse duration, the method and apparatus of laser thrombolysis can achieve sufficient ablation. The time to lyse a thrombus is given by the ablation efficiency is about 2 mg/mJ more or less independent of the pulse duration. Ablation efficiency is the mass in grams of tissue removed by the laser per energy of laser pulse used.

The user can then select the thrombus laser source 314 to emit a pulsed laser light energy. In accordance with one exemplary protocol, the thrombus laser source 314 can be in the green spectrum of optical energy, approximately 532 nanometers and a pulse duration of about 200 microseconds. The pulsed laser green light is incident on the thrombus, absorbed and causes a temperature increase leading to vapor formation and lysing of the thrombolytic material. Higher temperature increased can also be achieved. For example, temperatures increases up to and greater than 65 degrees C. can be achieved. Different wavelengths of light can be used to identify and heat the red, white and mixed thrombi. Wavelength sensitivity of different types of thrombus can also enhance the specificity of lysing red, white and mixed thrombi. Additionally, the user may select the power, pulse, and wavelength of the laser depending on the stage of the thrombosis. Higher power or an increase in frequency may be needed for late stage thrombosis, while lower power and frequency may be appropriate for early stage thrombosis.

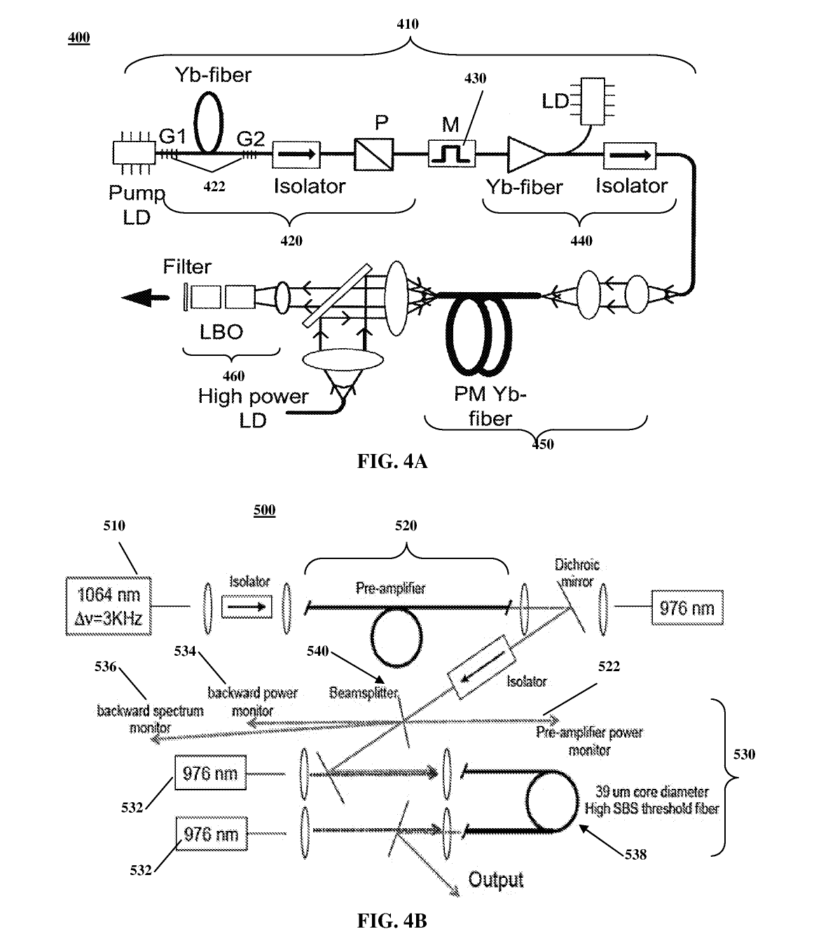

The thrombus laser beam may be derived from various pulsed laser light sources include q-switched, free-running, intracavity frequency doubled lasers, femtosecond lasers, diode pumped fiber lasers, UV excimer lasers and the like. In one embodiment, apparatus combines novel diode pumped fiber lasers that can produce diffraction limited (M²<1.5) high energy pulses (mJ) with a 100 μsec to 10 msec pulse duration that are absorbed by the thrombus material, such as hemoglobin and platelets. The diode pumped fiber laser sources are unique and ideally suited for OCT guided laser thrombolysis as they can provide diffraction limited ablative laser pulses of the appropriate pulse duration (100 μsec-100 msec), energy (5-20 mJ), and wavelength (400-1000 nm). A wavelength in the 350-600 nm regions is selected for laser thrombolysis to selectively ablate red and pink thrombus without incurring injury to the vessel wall. An exemplary diode pumped fiber laser 400 is shown in FIG. 4A. A high power Yb-doped laser 410 serve as a seed laser for the green laser. The high-power Yb-doped laser 410 provides high CW power that can be modulated at any desired pulse duration and repetition rate, and provides a beam that is nearly diffraction limited for coupling into a single mode OCT fiber. The diode pumped fiber laser 400 generally comprises a modulator, diode-pumped amplifiers, and non-linear conversion of the light using either SHG or DFG using various types of non-liner optical crystals such as Lithium Triborate LiB₃O₅ (LBO), Potassium Titanium Oxide Phosphate, KTiOPO₄ (KTP), Periodically poled Lithium Niobate, LiNbO₃ (PPLN), and the like to provide for frequency doubling. The non-linear conversion of the light to another wavelength can be done by the various non-linear optical materials through the physical processes of SHG (second harmonic generation) or possibly DFG (difference frequency generation). DFG requires the input of two wavelengths to get a third. The diode-pumped fiber laser is then modulated, amplified (with a diode pumped fiber amplifier) and frequency converted (e.g., doubled) to the wavelength of interest.

Exemplary Yb-Doped Fiber Laser

The Yb-doped fiber laser 410 as shown in FIG. 4A, with an Yb-doped Large-Mode-Area (“LMA”) fiber, the fiber-optic power amplifiers (“FOPA”) has generated as much as 2.4 kW of peak power without the onset of nonlinear effects. The diffraction-limited beam quality from the FOPA allows Potassium-titanyl-phosphate (“KTP”) or periodically poled (“PPKTP”) crystals to be replaced with Lithium Triborate (“LBO”) crystals to increase the interaction length to achieve efficient second-harmonic generation (“SHG”) without gray tracking problems. The green laser 400 based on frequency doubling of the FOPA consists of a continuous-wave (“cw”) fiber oscillator 420, an amplitude modulator (M) 430, a fiber preamplifier 440, a fiber power amplifier 450, and a second-harmonic generator 460, as shown schematically in FIG. 4A. The cw Yb-doped fiber oscillator 420 includes two fiber Bragg gratings (G1, G2) 422 generates a narrow linewidth at 1080 nm. The laser linewidth was measured to be less than 20 pm. The cw laser light is then modulated by the amplitude modulator 430, which can vary the pulse duration and the repetition rate independently. The pulse duration of the seed source can be varied from hundreds of microseconds to nanoseconds, and the repetition rate can be varied from hundreds of kilohertz to hundreds of megahertz. The average output power after the modulator is determined by the modulation duty factor. At a 10-MHz repetition rate (100-ns repetition period) and 5-ns pulse duration, a duty factor of 0.05 produces approximately 1 mW of average signal power. The signal is then amplified by the single-mode Yb-doped fiber preamplifier 440. The maximum output power from the preamplifier 440 is 200 mW, and the maximum gain is 23 dB. Fiber isolators are used between the fiber oscillator 420, the preamplifier 440, and the fiber power amplifier stages to protect each stage from backreflection, which can affect the performance of each stage. As the ratio of pulse duration to repetition period varies, the seed source provides a range of peak powers, and the preamplifier has reached a peak power of tens of watts without the onset of nonlinear effects. The fiber power amplifier 450 uses an Yb-doped polarization-maintaining double-clad LMA fiber with a fundamental mode-field diameter of 18 mm and a numerical aperture of 0.06.

An exemplary FOPA or masteroscillator power amplifier (“MOPA”) 500 is shown in FIG. 4B. A high power MOPA 500 may be constructed using a fiber manufactured according to the design of with a graded alumina and germania dopant profile in the core. The fiber may include a core diameter of 39 a hexagonal inner cladding diameter of 420 μm and an outer cladding diameter of 520 μm. The measured numerical apertures of the core and inner cladding may be 0.05 and 0.30, respectively. The Yb₂O₃ concentration may be increased to 1 wt-% to allow for a shorter fiber to be used. The pump absorption in the inner cladding of the fiber may be 3.2 dB/m at 976 nm. A schematic of the MOPA constructed using this fiber is illustrated in FIG. 4B. The MOPA 500 includes a signal source 510, which is a commercially available fiber laser generating 100 milliWatts of power at a wavelength of 1064 nm with a spectral width of 3 KHz. The output from the signal source 510 is amplified to a power level of 5 Watts in a pre-amplifier 520 comprising 4 meters of a conventional polarization maintaining Yb-doped double clad fiber with a core diameter of 20 μm. This fiber is coiled to a diameter of 70 mm to remove higher order modes resulting in an M2 value of 1.06 at the pre-amplifier output. The output of the pre-amplifier 520 is then launched into a power amplifier stage 530 constructed using the high SBS threshold fiber 538, as described above. This fiber is pumped bidirectionally with fiber coupled laser diode stacks 532. Each pump source 532 is capable of delivering up to 400 Watts of power at a center wavelength of 976 nm in a 400 um core, 0.22 NA fiber. A beam splitter 540, comprising an anti-reflection coated wedge with 0.3% reflectivity per surface, is placed between the two amplifier stages. This provides monitoring of the output power 522 from the pre-amplifier 520 and also of backward propagating light 534, 536 from the power amplifier stage 530. The optical spectrum 536 and average power 534 of the backward light are monitored continuously to observe the onset of stimulated Brillion scattering. Each stage of amplification is separated by >60 dB of isolation, which suppresses parasitic oscillations in the amplifier system that is capable of damaging the output end of the power amplifier stage. The MOPA 500 has a high power operation of narrow linewidth optical fiber amplifiers over 500 Watts of power in a single mode beam from a fiber designed to suppress stimulated Brillouin scattering through a reduction in the overlap of the optical and acoustic fields. The MOPA achieves greater than 1000 Watts of output power.

In order to couple efficiently light into a single-mode optical fiber such as those utilized in OCT intravascular imaging systems, a high beam quality is desired. The diode pumped fiber laser source provides a near diffraction limited beam quality at the fiber output. If only the fiber core diameter and the numerical aperture are known, and a step-index multimode fiber is assumed. There is no formula to exactly calculate the beam quality in that case, because it depends on the distribution of optical power over the fiber modes, and this distribution itself depends on the launching conditions. However, the beam quality M² factor can be roughly estimated, assuming that the power is well distributed over the modes, so that the numerical aperture represents a reasonable (perhaps slightly too high) estimate for the actual beam divergence. This leads to the Equation (4):

$${M}^{2} \approx \frac{Π a}{λ} NA ; \qquad ( 4 )$$

where α is the fiber core radius (i.e., half the core diameter). Such power dimensions should be accounted for to couple efficiently light from the thrombus laser into the optical fiber used in the OCT system. M² factors near unity correspond to diffraction limited beams. Use of large mode area fiber lasers and amplifiers (Yb fiber lasers and amplifiers) allows producing near diffraction limited beam quality of the thrombus laser light that can then be coupled efficiently into a single mode optical fiber such as that used in an intravascular OCT imaging system.

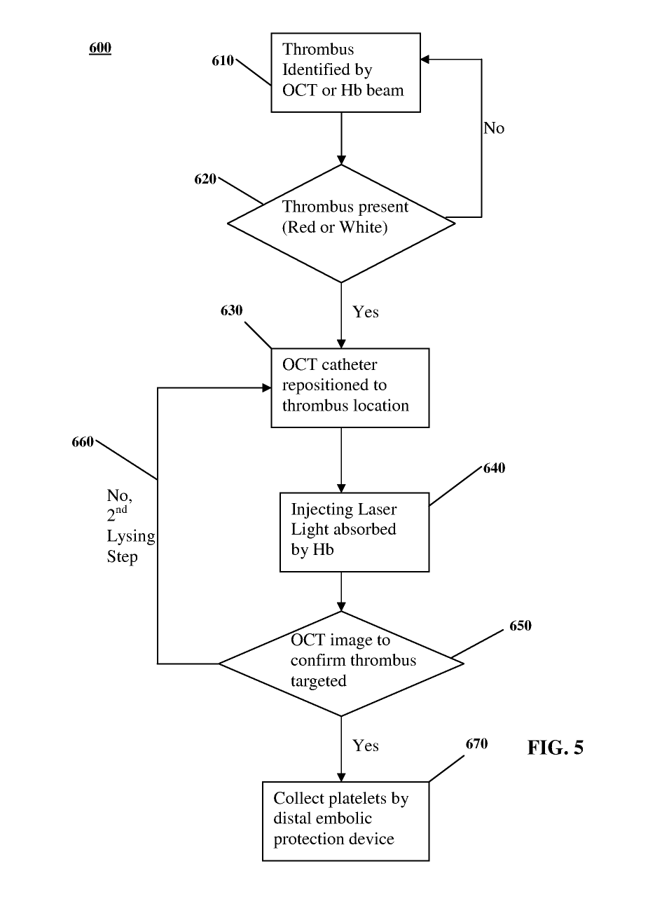

The method of simultaneous visualization of the intravascular space while conducting laser thrombolysis 600 comprises performing an intravascular OCT pullback image of a vessel being interrogated, generally shown as a flowchart in FIG. 5. If a thrombus is identified by OCT or by a hemoglobin beam 610, a next step 620 is completed to determine if the thrombus is red, which contains less reflectivity of Hemoglobin wavelengths, or if the thrombus is white, which contains more fibrin and has more Hemoglobin reflectivity. Red thrombi may be targeted for lysis by injecting laser light (e.g., 532 nm) that is absorbed by the hemoglobin in the thrombus. Similarly, white thrombi may be targeted by a laser that is absorbed by the constituents of white thrombi, fibrin, hemoglobin, platelets, etc. The identification of wavelengths for the white thrombi that are not absorbed by the contrast may then be made. The OCT catheter may then be repositioned to the thrombus location 630 by the pullback of the OCT catheter. The pullback of the OCT catheter may be in the range of 0.1 mm/s to 10 mm/s depending on the likelihood of the vessel being thrombotic, vessel tortuousness, size, and the like. After the OCT image pullback is completed and/or a thrombus is identified (either by the physician or using a feature identification algorithm), the OCT catheter is repositioned to a longitudinal location along the vessel to target the thrombus by the OCT image pullback. A second OCT image may be recorded to confirm that light exiting the OCT catheter is targeting the thrombus. After identification of the thrombus and positioning of the OCT catheter for targeting the thrombus is complete, the instrument is configured in a thrombolysis operating mode to inject laser light absorbed by hemoglobin 640. In the thrombolysis operating mode, a number of parameters are fixed, these include: (1) rotational speed of the catheter (revs per sec); (2) pulse repetition rate of the thrombolysis laser (pulses per second); (3) pulse duration of each laser pulse (microseconds or milliseconds); (4) energy per pulse (mJ); (5) pullback speed of the catheter while lysing the thrombus (mm/sec); (6) duration of laser exposure (sec); (7) flushing parameters (medium for flushing, volume of flush (ml), flow rate (ml/sec). With the selection of the parameters associated with the thrombolysis operating mode, the procedure to lyse the thrombus is completed. After completion of a thrombus lysing procedure, the region may be imaged using OCT to confirm the lysing of the thrombus 650. If the thrombus is incompletely lysed, the physician may choose to execute a second lysing procedure 660. The lysed thrombi may be collected by a distal embolic protection device 670.

Alternatively, the laser thrombus method and apparatus can be combined with a distal embolic protection device 390, generally shown in FIG. 3C. The distal embolic protection device would collect or filter out particles which will be dislodged by the laser ablation and prevent them from entering the pulmonary system or other sensitive arteries and vessels. The distal embolic protection device would generally allow for the flushing of the vessel to be undeterred, but catch the dislodged thrombosis distal to the catheter tip.

The method and apparatus comprise a combined action of an intravascular OCT system with a thrombus laser with nearly diffraction limited (M²<1.5), high average power (hundreds of watts), high repetition rate, long pulse duration, high pulse energy laser sources for thrombolysis. At the back-end of the system, there could be a software modification or imaging processes to overlay the image of the OCT thrombus image before thrombolysis and after thrombolysis.

Differential Detection, Staging and Treatment of Intravascular Thrombus

The present system and method is well suited to detect and intravascular thrombus in the arterial or the venous system, including within both high pressure pulsatile flow vessels such as the coronary arteries and within low pressure non-pulsatile flow vessels such in leg veins or venous grafts. A primary trigger for arterial thrombosis is the rupture of an atherosclerotic plaque, which develops through the accumulation of lipid deposits and lipid-laden macrophages (foam cells) in the artery wall. The thrombi that form at ruptured plaques are rich in platelets, which are small (about 1 μm in diameter) anucleate cells produced by megakaryocytes in the bone marrow. These disc-shaped cells circulate in the blood as sentinels of vascular integrity and rapidly form a primary haemostatic plug at sites of vascular injury. When an atherosclerotic plaque ruptures, platelets are rapidly recruited to the site, through the interaction of specific platelet cell-surface receptors with collagen and von Willebrand factor. After this adhesion to the vessel wall, the receptor-mediated binding of additional platelets (termed platelet aggregation) then results in rapid growth of the thrombus. Platelets also become activated at this stage. A major pathway of activation involves the cleavage and, consequently, the activation of the platelet receptor PARI (protease-activated receptor 1; also known as the thrombin receptor) by the protease thrombin (also known as factor II), which is activated by the blood coagulation cascade. Activated platelets then release the contents of granules, which further promote platelet recruitment, adhesion, aggregation and activation.

The coagulation cascade is the sequential process by which coagulation factors of the blood interact and are activated, ultimately generating fibrin, the main protein component of the thrombus, and this cascade operates in both arterial and venous thrombosis. The cascade is initiated by exposure of the blood to tissue factor (also known as factor III), a protein that is present at high concentrations in atherosclerotic plaques. Circulating tissue factor is also present at increased concentrations in patients with cardiovascular disease and might contribute to thrombosis after plaque rupture.

Venous thrombosis can be triggered by several factors, including abnormal blood flow, e.g., the absence of blood flow; altered properties of the blood, e.g., thrombophilia, or alterations in the endothelium. In venous thrombosis, the endothelium typically remains intact, but is converted from a surface having anticoagulant properties to one with procoagulant properties.

Thrombus staging may be accomplished by the OCT system and method of the present invention in which the differential cellular characteristics of disorganized early stage thrombus are employed to discriminate from the more highly organized later stage thrombus. It is generally understood that earlier stage thrombus is formed proximally on a lesion relative to the later stage thrombus. Additionally, disorganized early stage thrombus is more susceptible to thrombolysis using lower energy or pulsed laser energy than that required to lyse later stage thrombolysis.

Thus, an initial OCT imaging pass may be made to identify and stage the thrombus lesion as being early stage, later stage or a combination thereof. Once the staging information is obtained and the position of the respective staged thrombus determined, either the power of or the pulse rate of the Thrombus Laser is adjusted to correlate to the type of thrombus and applied to the thrombus. In one embodiment of the inventive method, the power to the Thrombus Laser is reduced to lyse the earlier stage thrombus and clear it from the lesion. In another embodiment of the inventive method, the Thrombus Laser is pulsed at a rate to differentially lyse the early stage thrombus and clear it from the lesion. In yet another embodiment of the inventive method, the power and/or pulse rate is then increased to lyse the later stage, more highly organized thrombus.

IVUS/OCT Catheter

Additionally, a combined IVUS/OCT catheter may first identify suspicious thrombi regions with Intravascular Ultrasound (“IVUS”). An exemplary IVUS/OCT catheter is readily understood by nonprovisional application entitled “OCT-IVUS Catheter for Concurrent Luminal Imaging”, U.S. Ser. No. 12/173,004, filed Jul. 14, 2008. In a first step, the thrombus is identified and imaged with IVUS. The elastic properties of the thrombus can also be estimated to determine if the thrombus is soft or rigid. In a second step, a limited volume contrast injection is used and the thrombus can be imaged with OCT. After imaging the thrombus with OCT and making a determination of the thrombus color (white or red), the thrombus laser may be applied to break the thrombus into small fragments for removal. The procedure is: (1) first image with IVUS; (2) image with OCT; (3) identify thrombus type; and (4) perform laser thrombolysis.

The combined IVUS/OCT catheter is able detect the mechanical properties of the thrombus using IVUS elasticity imaging. The mechanical properties of thrombus change with time and the thrombi become more rigid over time. Elasticity imaging of thrombi to detect the mechanical properties of the thrombus examines how the thrombus responds to the flush in the OCT image. Rigid thrombus do not show surface distortion and tend to displace more readily as opposed to soft thrombus, which would indicate more surface distortion and less gross displacement in response to a flush. To differentiate between soft and rigid thrombus the examination of how the thrombus responds to the momentum of the flush material—if the response is greater surface displacement and less gross movement or movement of the center of mass, then that indicates soft thrombus. While if the thrombus surface does not distort and there is gross displacement, which indicates rigid thrombus.

Ultrasound elasticity imaging to noninvasively detect and age thrombus knowing that thrombi harden over time is useful, but the technique relies on whether the age of a thrombus can be predicted from strain estimates, and how accurate these predictions are. Thrombus hardness can be quantified at each scan interval by measures of normalized strains and reconstructed relative Young's moduli. Strain magnitudes exhibit progressive decrease as clots age and the relationship between the normalized strain and the clot age can be developed. Elasticity imaging is a key component of venous compression ultrasound for effective diagnosis and treatment of thrombosis.

Auto-Initiation and Flushing

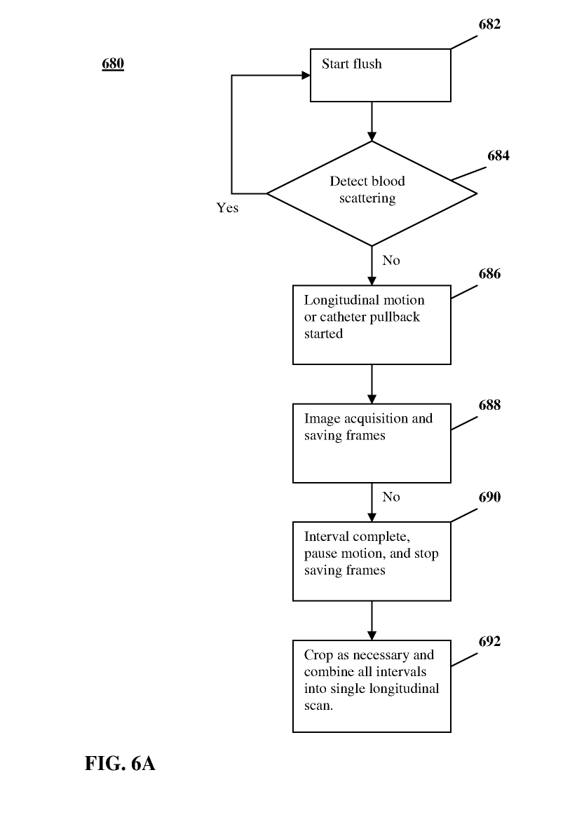

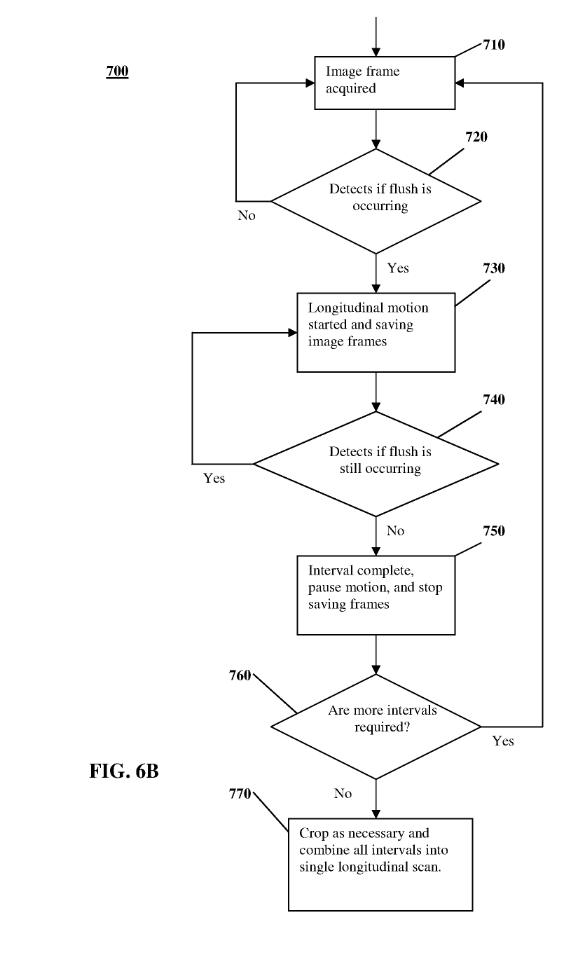

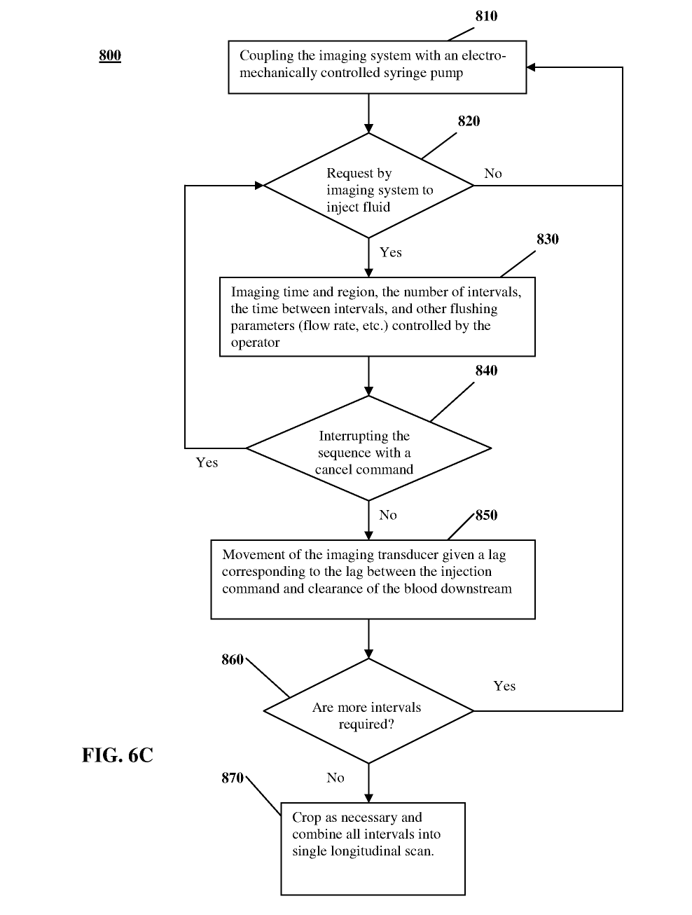

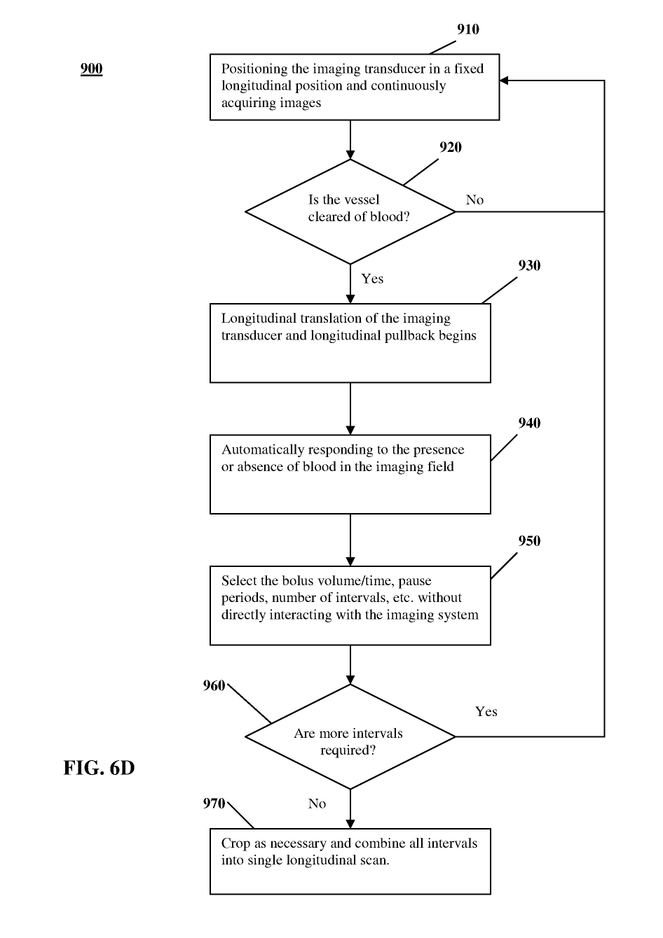

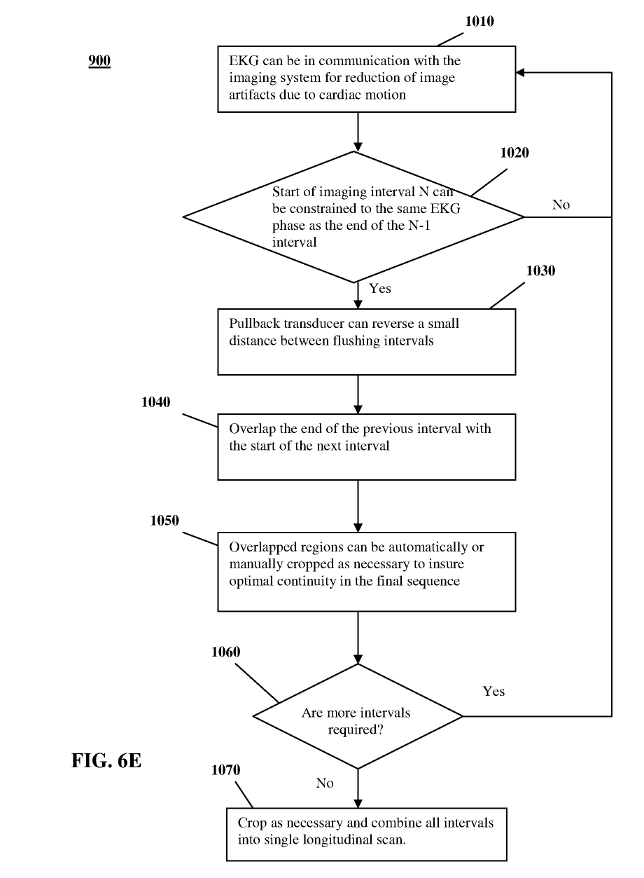

Blood backscattering or erythrocyte backscattering can be employed to detect the start and the stop of a flush in the vessel lumen during imaging. The blood backscattering or hemoglobin reflectivity measurement/signal is at a maximum during periods before and after the saline flush and drops substantially when the flush bolus arrives; thus represents a signal change. Blood backscattering or hemoglobin reflectivity measurement may be accomplished by optical energy, sound energy, radiofrequency, magnetic or nuclear energy, and the like. As shown in FIG. 6A, detecting this dramatic signal change is a start signal for OCT longitudinal motion or the pullback device of the OCT catheter system by a method 680. Alternatively, the detection of the signal change for blood backscattering may be employed with an alternative imaging system, including by way of example and not limitation, spectroscopic devices, (including fluorescence, absorption, scattering, and Raman spectroscopies), intravascular ultrasound devices (IVUS), Forward-Looking IVUS (FLIVUS) devices, high intensity focused ultrasound (HIFU), radiofrequency, thermal imaging or thermography, optical light-based imaging, magnetic resonance, radiography, nuclear imaging, photoacoustic imaging, electrical impedance tomography, elastography, pressure sensing wires, intracardiac echocardiography (ICE), forward looking ICE and orthopedic, spinal imaging and neurological imaging, image guided therapeutic devices or therapeutic delivery devices, diagnostic delivery devices, and the like. The automatic detection of flushing may be communicated to computer, operator, or physician and the subsequent activation of the longitudinal motion of the catheter pull back may be automatic or initiated by an operator or physician. All steps and processes below may be executed by a computer program, computer, electric-mechanical system, operator, physician, and the like.

Generally, the method 680 comprises starting a flushing sequence 682 in a catheter. The flush sequence may be started by a flushing apparatus operably coupled to the distal end of the catheter. Then, decision 684 detects if blood scattering is present. The detection of the blood scattering may be employed by an imaging system operably coupled to the distal end of the catheter by a wire, optical fiber, and the like. The detection signal may be displayed by a computer system operable coupled to the imaging system. If blood scattering is not present, then step 686 initiates the longitudinal motion or pullback of the catheter. The longitudinal motion or pullback of the catheter is employed with a longitudinal displacement apparatus operably coupled to the proximal end of the catheter. In one embodiment, the longitudinal displacement apparatus is the Volcano™ Revolution™ PIM, the Volcano™ R100, or the Volcano™ Trak Back II Catheter Pull-Back Device. The longitudinal displacement apparatus may be operably coupled to the computer system. In one embodiment, the start signal for longitudinal motion or pullback of the catheter device is initiated by the detection of the blood scattering 684. The start signal may be sent from a computer component operably coupled to the imaging system and the longitudinal motion apparatus or from a user operated signal. Alternatively, the start signal for longitudinal motion is initiated by the image detection of blood scattering 684. The image detection of blood scattering may be employed on a display device operably coupled to the imaging system. Then, step 688 starts the image acquisition and saving of the image frames to a memory device operably coupled to the imaging system or computer system. Step 690 determines if the imaging interval is complete, at which point the longitudinal motion or pullback is paused and the saving of image frames is paused. The imaging interval may be predetermined or manually entered or operated by a user or physician. If no more images are required, the step 692 crops the images as necessary and combines all intervals into single longitudinal scan. If more images are required, method 680 is repeated as necessary by the user or a preselected option for number of images to be acquired.

Different pullback distances and pullback rates may be utilized for varying flush sequences, altered for detection of blood scattering during the pull back sequence, or for varied anatomical vessels to be imaged. In at least some embodiments, the pullback distance of the catheter is at least between 0.01 mm and 100 cm. In at least some embodiments, the pullback distance of the imaging core is at least between 10 mm and 10 cm. In at least some embodiments, the pullback distance of the imaging core is at least between 15 mm and 15 cm. In at least some embodiments, the pullback distance of the imaging core is at least between 20 mm and 20 cm. In at least some embodiments, the pullback distance of the imaging core is at least between 25 mm and 25 cm. In some embodiments, the linear pullback rate is along the portion of the vasculature and has a linear pullback rate in the range of 0.01 mm/sec to 100 cm/sec. In at least some embodiments, the region of interest is imaged using a linear pullback rate of no less than 2 mm/sec. In at least some embodiments, the region of interest is imaged using a linear pullback rate of no less than 10 mm/sec. In at least some embodiments, the region of interest is imaged using a linear pullback rate of no less than 50 mm/sec. In at least some embodiments, the region of interest is imaged using a linear pullback rate of no less than 75 mm/sec. In at least some embodiments, the region of interest is imaged using a linear pullback rate of no less than 90 mm/sec. In at least some embodiments, the region of interest is imaged using a linear pullback rate of no less than 30 mm/sec. In at least some embodiments, the region of interest is imaged using a linear pullback rate of no less than 40 mm/sec. In at least some embodiments, the region of interest is imaged using a linear pullback rate between about 40-100 mm/sec. Alternatively, the pullback rate may also be nonlinear, exponential, and the like.

One embodiment of the method for detecting the start and stop of a flush during imaging 700 comprises reducing the total volume-per-bolus of blood-clearing fluid delivered during intravascular imaging step, generally shown in FIG. 6B. The method may be generally operated by a computer or processor coupled to the imaging system, whereby the imaging system is an OCT imaging system, IVUS, spectroscopy, light, or other imaging system as described previously. An initial flushing step and an image frame is acquired at step 710 by the imaging system operably coupled to the distal end of the catheter, and step 720 detects if flushing is occurring 720. In one embodiment, an algorithm may detect if flushing is occurring, which may either be the detection of the backscattering of the flushing fluid, the reflectivity of the flushing fluid, or the image detection of the flushing fluid. If flushing is occurring or detected, step 730 commences the longitudinal motion of the catheter by the longitudinal displacement apparatus and the image frames are saved to a memory device operably coupled to the imaging system. Step 740 detects if flushing is still occurring past the initial flushing step, and if no flushing is occurring then the interval is complete and the longitudinal motion and image frame saving is stopped at step 750. The flushing stop signal is sent to the flushing apparatus operably coupled to the distal end of the catheter. The detection of the flushing may be accomplished by algorithm and the like, as previously indicated. If flushing is still occurring, then step 740 is repeated until flushing is stopped and no longer detected. If more imaging intervals are required at decision 760, the method proceeds to step 710 to acquire an image frame with the imaging system. If more intervals are not required at decision 760, then the method proceeds to step 770 to crop the image frames as necessary and combine all intervals into a single longitudinal scan. The total imaging time and region is subdivided into two or more separate intervals, each of which is imaged serially in time with pauses between each interval. The pauses between each interval allow for reflow of blood, which allows the effective ischemic load of the flushing to be spread out over a longer and safer time period. The rate and distance between each pause may be varied according any longitudinal distance or pullback rate. Two different methods for interval-based imaging are below.

Another embodiment of the method for detecting the start and stop of a flush during imaging 800 is shown in FIG. 6C and generally comprises step 810 of coupling the imaging system to be in communication with an electro-mechanically controlled syringe pump, a “power flusher”, or any other electrical flushing apparatus. The flushing apparatus includes a syringe reservoir that is pre-loaded with clearing fluid and injects the clearing fluid at decision 820 only as requested by the imaging system. The request may be entered by a user or automated by the computer system and some predetermined initiation. At step 830, the total imaging time and region, the number of intervals, the time between intervals, and other flushing parameters (flow rate, fluid pressure, fluid volume, etc.) are controlled by the operator at the physician's discretion or by preselected parameters entered into the flushing apparatus. A single command starts the sequence, and the control of the flushing and the imaging location on the distal end of the catheter is automated by the imaging system. At step 840, a cancel command is available to the operator for interrupting the sequence once it is started. Because a lag time may exist between the injection command and clearance of the blood downstream, step 850 provides for the movement of the imaging transducer to be given a corresponding lag time. The movement of the imaging transducer may be any rate or distance as to correspond to the lag time. The lag time can be a function of the transducer position relative to the distal end of the guide catheter or where the fluid is delivered. Decision 860 decides if more intervals are required. If no other intervals are required, Step 870 crops the images as necessary and combines all the intervals in a single longitudinal scan, and stops the flushing apparatus. If more intervals are required, then step 810 ensures that the imaging system is still coupled with the electro-mechanically controlled syringe pump to proceed with method 800 for an additional imagine interval.

Another embodiment of the method for detecting the start and stop of a flush during catheter imaging 900 is shown in FIG. 6D and generally comprises step 910 of positioning the imaging transducer in a fixed longitudinal position and continuously acquiring images with the imaging system. Generally, the imaging transducer is positioned on the distal end of the catheter; however, the imaging transducer may be positioned on any portion of the catheter in relation to the flush entry point on the catheter. Decision 920 analyses the incoming images and determines if the vessel is cleared or not cleared of blood, as indicated previously. In one embodiment, a real-time image processing algorithm analyses the incoming images and determines if the vessel is cleared or not cleared of blood. Alternatively, the backscattering of the blood may be detected to determine if the vessel is cleared or not cleared of blood. If the vessel is cleared of blood, then step 930 proceeds with the longitudinal translation of the imaging transducer and longitudinal pullback of the catheter begins with the longitudinal motion apparatus operably coupled to the catheter. In one embodiment, only during intervals when the algorithm detects a cleared field (blood not present) is the longitudinal translation of the imaging transducer initiated and longitudinal pullback begins. If during imaging intervals, a cleared field is not detected or blood backscattering is detected, the longitudinal pullback may be paused for further flushing. If any interval is interrupted, increased pullback rates may be used to prevent prolonged imaging periods. Then, step 940 automatically responds to the presence or absence of blood in the imaging field with the imaging system. Step 950 allows the physician or operator to start flushing and select the flushing parameters, such as the bolus volume/time, pause periods, number of intervals, etc. without directly interacting with the imaging system. Alternatively, preselected start flushing sequences and parameters maybe initiated by a computer system. Decision 960 decides if more intervals are required. If additional intervals are required, alternative pullback rates may be used to prevent prolonged imaging periods. If no other intervals are required, step 970 crops the images as necessary and combines all the intervals in a single longitudinal scan. If further image intervals are required, then step 910 ensures that the positioning the imaging transducer in a fixed longitudinal position for further continuously acquiring images.

Finally, for any method used for generating the flush intervals, images of non-flushed vessel at the start and end of each interval can be cropped out and all intervals combined into what appears to the user as a single image sequence of the total region of interest. In one embodiment, an Electrocardiography (EKG) can be in communication with the imaging system for reduction of image artifacts due to cardiac motion in step 1010, as shown in FIG. 6E. Electrocardiography is a transthoracic interpretation of the electrical activity of the heart over time captured and externally recorded by skin electrodes. An EKG apparatus may be in communication with the imaging system via optical, electrical, wireless, or any other communication device. In one embodiment, at step 1020 the start of imaging interval N can be constrained to the same EKG phase as the end of the N−1 interval, effectively making the image sequence appear more continuous. At step 1030, the pullback transducer can reverse a small distance between flushing intervals and intentionally overlap the end of the previous interval with the start of the next interval at step 1040. Catheter distances and pull back rates may be employed for the constrained EKG phase. Overlapped regions can be automatically or manually cropped as necessary to insure optimal continuity in the final sequence at step 1050.

EKG synchronization to alleviate registration artifacts due to the catheter sliding longitudinally back and forth during heart beat motion may be coupled with any of the methods 600, 700, 800, and 900 previously described. In one embodiment, if the thrombus is imaged first but then lysed on a second pull-back, the lysing beam should only fire at the same point in the EKG at which the image was acquired (or the pullbacks should be started at the same phase in the EKG).

It will be understood that each block of the flowchart illustrations, and combinations of blocks in the flowchart illustrations, as well any portion of the tissue classifier, imager, control module, systems and methods disclosed herein, can be implemented by computer program instructions. These program instructions may be provided to a processor to produce a machine, such that the instructions, which execute on the processor, create means for implementing the actions specified in the flowchart block or blocks or described for the tissue classifier, imager, control module, systems and methods disclosed herein. The computer program instructions may be executed by a processor to cause a series of operational steps to be performed by the processor to produce a computer implemented process. The computer program instructions may also cause at least some of the operational steps to be performed in parallel. Moreover, some of the steps may also be performed across more than one processor, such as might arise in a multi-processor computer system. In addition, one or more processes may also be performed concurrently with other processes or even in a different sequence than illustrated without departing from the scope or spirit of the invention.

The computer program instructions can be, stored on any suitable computer-readable medium including, but not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computing device.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

What is claimed is:

1. A method for starting the longitudinal motion of a catheter system, comprising:
starting a flushing sequence;
detecting if blood is present; and
initiating the longitudinal motion of the catheter.

2. The method of claim 1, further comprising initiating an imaging device for image acquisition.

3. The method of claim 2, wherein the detecting step further comprises detecting blood scattering.

4. The method of claim 2, wherein the detecting step further comprises detecting an image of blood.

5. The method of claim 2, further comprising saving the image frames to a memory device.

6. The method of claim 5, further comprising determining if an image interval is complete, and pausing the longitudinal motion of the catheter.

7. The method of claim 6, further comprising cropping the images and combining all image into single longitudinal scan.

8. A method for detecting the start and stop of a flush during a catheter imaging procedure comprising:
reducing the total volume-per-bolus of blood-clearing fluid delivered during an imaging step;
acquiring an image frame; and
detecting if flushing is occurring.

9. The method of claim 8, further comprising communicating if flushing is occurring to an operator and initiating the longitudinal motion of the catheter pull back by the operator.

10. The method of claim 8, wherein the detecting step further comprises commencing longitudinal motion of the catheter if flushing is occurring; and completing the image interval if no flushing is occurring and stopping the longitudinal motion of the catheter.

11. The method of claim 10, further comprising detecting if flushing is still occurring, and checking to detect if flushing is occurring until flushing is stopped.

12. The method of claim 11, further comprising determining if more image intervals are required, and acquiring the image frame.

13. The method of claim 11, further comprising cropping the frames and combining the interval images into a single longitudinal scan if more image intervals are not required.

14. The method of claim 13, further comprising subdividing the total imaging time and region into two or more separate intervals, wherein each image is imaged serially in time with at least one pause between each interval.

15. The method of claim 14, wherein the at least one pause between each interval allows for the reflow of blood through the vessel.

16. The method of claim 15, wherein a computer processor saves the image frames.

17. A method for detecting the start and stop of a flush during a catheter imaging procedure comprising:
coupling an imaging system in communication with an electro-mechanically controlled syringe pump;
pre-loading the syringe reservoir with a clearing fluid; and
injecting the clearing fluid into the distal end of a catheter only as requested by the imaging system.

18. The method of claim 17, further comprising acquiring images with the imaging system, wherein at least one of the total imaging time and region, the number of intervals, the time between intervals, and other flushing parameters are controlled by the operator.

19. The method of claim 18, wherein a single command starts the sequence, and the control of the flushing and imaging location are automated by the imaging system.

20. The method of claim 19, further comprising interrupting the imaging sequence with a cancel command.

21. The method of claim 20, further comprising providing a lag between the movement of the imaging transducer and acquiring images corresponds with a lag between the injection command and the clearance of the blood.

22. The method of claim 21, wherein the lag time is a function of the imaging transducer position relative to the distal end of the catheter.

23. A method for detecting the start and stop of a flush during a catheter imaging procedure comprising:
positioning an imaging transducer in a fixed longitudinal position and continuously acquiring images with an imaging system;
analyzing the incoming images and determining if the vessel is cleared or not cleared of blood; and
initiating the longitudinal translation of the imaging transducer when a cleared vessel is detected.

24. The method of claim 23, further comprising selecting the bolus volume/time, pause periods or the number of intervals, without directly interacting with the imaging system.

25. The method of claim 24, further comprising cropping out images of the non-flushed vessel and combining all intervals as a single image sequence for a region of interest.

26. The method of claim 25, further comprising constraining the start of the imaging interval-N to the same EKG phase as the end of the N+1 interval to make the image sequence continuous.

27. The method of claim 26, further comprising reversing the pullback transducer between the flushing intervals to overlap the end of the previous interval with the start of next interval to crop the imaged region.

28. The method of claim 27, further comprising synchronizing the image intervals to the EKG to alleviate registration artifacts due to the catheter sliding longitudinally back and forth during the heart beat motion.

29. The method of claim 2, wherein the imaging system is selected from a group consisting of an Optical Coherence Tomography imaging system, a spectroscopic device, an intravascular ultrasound (IVUS) device, a Forward-Looking IVUS (FLIVUS) device, a high intensity focused ultrasound (HIFU) device, a radiofrequency device, a thermal imaging or thermography device, an optical light-based imaging device, a magnetic resonance device, a radiography device, a nuclear imaging device, a photoacoustic imaging device, an electrical impedance tomography device, an elastography device, a pressure sensing wire device, an intracardiac echocardiography (ICE) device, a forward looking ICE device, an orthopedic device, a spinal imaging device, a neurological imaging device, an image guided therapeutic device, a therapeutic delivery device, and a diagnostic delivery device.

30. A catheter system for the longitudinal motion of the catheter and image initiation, comprising:
a. a flushing apparatus operably coupled to the distal end of a catheter;
b. an imaging system operably coupled to the distal end of the catheter to detect if blood is present; and
c. a longitudinal displacement apparatus operably coupled to the catheter to initiate the longitudinal motion of the catheter upon the detection of blood.

31. The system of claim 30, wherein the imaging system is selected from a group consisting of an Optical Coherence Tomography imaging system, a spectroscopic device, an intravascular ultrasound (IVUS) device, a Forward-Looking IVUS (FLIVUS) device, a high intensity focused ultrasound (HIFU) device, a radiofrequency device, a thermal imaging or thermography device, an optical light-based imaging device, a magnetic resonance device, a radiography device, a nuclear imaging device, a photoacoustic imaging device, an electrical impedance tomography device, an elastography device, a pressure sensing wire device, an intracardiac echocardiography (ICE) device, a forward looking ICE device, an orthopedic device, a spinal imaging device, a neurological imaging device, an image guided therapeutic device, a therapeutic delivery device, and a diagnostic delivery device.

32. The catheter system of claim 30, wherein the flushing apparatus is operably associated with the imaging system and the flushing apparatus injects a clearing fluid only as requested by the imaging system.

33. The catheter system of claim 30, wherein the catheter further comprises an imaging transducer for acquiring images and the imaging transducer is in a fixed longitudinal position along the length of the catheter.

34. The catheter system of claim 30, wherein the flushing apparatus reduces the total volume-per-bolus of a flushing fluid delivered during an imaging step of the catheter.
